(19) United States
(12) Patent Application Publication
(10) Pub. No.: US 2025/0255461 A1
(43) Pub. Date: Aug. 14, 2025

(54) MEDICAL SUPPORT DEVICE, ENDOSCOPE SYSTEM, MEDICAL SUPPORT METHOD, AND PROGRAM

(71) Applicant: FUJIFILM CORPORATION, Tokyo (JP)

(72) Inventor: Misaki GOTO, Kanagawa (JP)

(21) Appl. No.: 18/977,935

(22) Filed: Dec. 12, 2024

(30) Foreign Application Priority Data
Feb. 9, 2024 (JP) 2024-018839

(51) Int. Cl. A61B 1/00; A61B 1/273

(52) U.S. Cl. CPC A61B 1/00045; A61B 1/000094; A61B 1/000096; A61B 1/273

(57) ABSTRACT

A medical support device includes a processor. The processor acquires a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope. The processor outputs support information for supporting the intubation in a case in which the difficulty level is equal to or higher than a reference difficulty level.

### CROSS-REFERENCE TO RELATED APPLICATIONS

This application claims priority under 35 USC 119 from Japanese Patent Application No. 2024-018839, filed on Feb. 9, 2024, the disclosure of which is incorporated by reference herein.

### BACKGROUND

### 1. Technical Field

The present disclosure relates to a medical support device, an endoscope system, a medical support method, and a program.

### 2. Related Art

JP2022-105685A discloses a technology of displaying an image from a camera on a flexible elongated member (see FIGS. 6A and 6B and FIGS. 6D and 6E) and executing detection and identification of a papilla for a medical worker. In addition, a controller described in JP2022-105685A determines a target trajectory based on an image of the papilla and provides a visual intubation path for the medical worker by superimposing the target trajectory on the image.

### SUMMARY

One embodiment according to the present disclosure provides a medical support device, an endoscope system, a medical support method, and a program that can support intubation into a duodenal papilla in a case in which a difficulty level of the intubation into the duodenal papilla is equal to or higher than a reference difficulty level.

A first aspect according to the present disclosure relates to a medical support device comprising: a processor, in which the processor acquires a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope, and outputs support information for supporting the intubation in a case in which the difficulty level is equal to or higher than a reference difficulty level.

A second aspect according to the present disclosure relates to the medical support device according to the first aspect, in which, in a case in which the difficulty level is determined based on the duodenal papilla, the difficulty level is determined based on an aspect of the duodenal papilla.

A third aspect according to the present disclosure relates to the medical support device according to the second aspect, in which the aspect of the duodenal papilla includes a shape of the duodenal papilla.

A fourth aspect according to the present disclosure relates to the medical support device according to the second or third aspect, in which the aspect of the duodenal papilla includes a size of the duodenal papilla.

A fifth aspect according to the present disclosure relates to the medical support device according to any one of the second to fourth aspects, in which the aspect of the duodenal papilla includes a type of an opening part of the duodenal papilla.

A sixth aspect according to the present disclosure relates to the medical support device according to any one of the second to fifth aspects, in which the aspect of the duodenal papilla includes a confluence form of a bile duct and a pancreatic duct in the duodenal papilla.

A seventh aspect according to the present disclosure relates to the medical support device according to any one of the second to sixth aspects, in which the aspect of the duodenal papilla includes a length of an oral side elevation.

An eighth aspect according to the present disclosure relates to the medical support device according to any one of the first to seventh aspects, in which, in a case in which the difficulty level is determined based on the peripheral region, the difficulty level is determined based on an aspect of the peripheral region.

A ninth aspect according to the present disclosure relates to the medical support device according to the eighth aspect, in which the aspect of the peripheral region includes presence or absence of a diverticulum.

A tenth aspect according to the present disclosure relates to the medical support device according to the eighth or ninth aspect, in which the aspect of the peripheral region includes deformation of the duodenum.

An eleventh aspect according to the present disclosure relates to the medical support device according to any one of the first to tenth aspects, in which the support information includes information indicating that the difficulty level is equal to or higher than the reference difficulty level.

A twelfth aspect according to the present disclosure relates to the medical support device according to any one of the first to eleventh aspects, in which the support information includes incision region specification information for specifying an incision region for incising the duodenal papilla.

A thirteenth aspect according to the present disclosure relates to the medical support device according to the twelfth aspect, in which the incision region is included in a region avoiding a blood vessel that is included in the duodenal papilla and that has an attention level equal to or higher than a certain attention level.

A fourteenth aspect according to the present disclosure relates to the medical support device according to the twelfth or thirteenth aspect, in which the incision region is predicted by executing image processing on the intestinal wall image, and the incision region specification information is a prediction result of the incision region obtained by the image processing.

A fifteenth aspect according to the present disclosure relates to the medical support device according to the fourteenth aspect, in which the image processing is processing of causing a first trained model to generate the prediction result by inputting the intestinal wall image to the first trained model.

A sixteenth aspect according to the present disclosure relates to the medical support device according to the fourteenth or fifteenth aspect, in which the intestinal wall image includes a first intestinal wall image in which a blood vessel that is included in the duodenal papilla and that has an attention level equal to or higher than a certain attention level is shown in a state of being specifiable, and the incision region is predicted to be within a region avoiding the blood vessel by executing the image processing on the first intestinal wall image.

A seventeenth aspect according to the present disclosure relates to the medical support device according to any one of the first to sixteenth aspects, in which the support information includes operation assistance information for assisting an operation of the endoscope.

An eighteenth aspect according to the present disclosure relates to the medical support device according to any one of the first to seventeenth aspects, in which the difficulty level is determined based on the duodenal papilla and/or the peripheral region recognized by executing recognition processing on the intestinal wall image.

A nineteenth aspect according to the present disclosure relates to the medical support device according to the eighteenth aspect, in which the recognition processing is processing of causing a second trained model to recognize the duodenal papilla and/or the peripheral region shown in the intestinal wall image by inputting the intestinal wall image to the second trained model.

A twentieth aspect according to the present disclosure relates to an endoscope system comprising: the medical support device according to any one of the first to nineteenth aspects; and the endoscope.

A twenty-first aspect according to the present disclosure relates to a medical support method comprising: acquiring a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope; and outputting support information for supporting the intubation in a case in which the difficulty level is equal to or higher than a reference difficulty level.

A twenty-second aspect according to the present disclosure relates to a program causing a computer to execute a medical support process comprising: acquiring a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope; and outputting support information for supporting the intubation in a case in which the difficulty level is equal to or higher than a reference difficulty level.

### BRIEF DESCRIPTION OF THE DRAWINGS

Exemplary embodiments of the technology of the disclosure will be described in detail based on the following figures, wherein:

FIG. 1 is a conceptual diagram showing an example of an aspect in which a duodenoscope system is used;

FIG. 2 is a conceptual diagram showing an example of an overall configuration of the duodenoscope system;

FIG. 3 is a block diagram showing an example of a hardware configuration of an electric system of the duodenoscope system;

FIG. 4 is a conceptual diagram showing an example of aspects of a duodenum, a bile duct, and a pancreatic duct;

FIG. 5 is a block diagram showing an example of main functions of a processor included in a medical support device and an example of information stored in a storage;

FIG. 6 is a conceptual diagram showing examples of recognition processing using a papilla recognition model executed by a recognition unit and display processing of an intestinal wall image executed by a controller;

FIG. 7 is a conceptual diagram showing an example of recognition processing using a difficulty level recognition model executed by the recognition unit;

FIG. 8 is a conceptual diagram showing an example of prediction processing using an insertion direction prediction model executed by the recognition unit;

FIG. 9 is a conceptual diagram showing an example of processing of acquiring support information from a support information table via the controller and processing of generating high difficulty level notification information via the controller;

FIG. 10 is a conceptual diagram showing an example of display processing using insertion direction information, support information, and the high difficulty level notification information executed by the controller;

FIG. 11A is a flowchart showing an example of a flow of a medical support process;

FIG. 11B is a continuation of the flowchart shown in FIG. 11A;

FIG. 12 is a conceptual diagram showing a modification example of the recognition processing using the difficulty level recognition model executed by the recognition unit;

FIG. 13 is a conceptual diagram showing an example of an aspect in which the high difficulty level notification information is generated based on a plurality of difficulty levels obtained by the recognition processing using the difficulty level recognition model shown in FIG. 12 and is displayed on a screen;

FIG. 14 is a conceptual diagram showing an example of processing contents of the controller in a case in which a difficulty level of intubation into a duodenal papilla is determined based on a confluence form and deformation of the duodenum;

FIG. 15 is a conceptual diagram showing an example of image processing using an incision region prediction model executed by the recognition unit;

FIG. 16 is a conceptual diagram showing an example of an aspect in which visible information (that is, an incision planned region mark) based on incision region specification information obtained by the image processing using the incision region prediction model executed by the recognition unit is displayed in a superimposed manner on the intestinal wall image displayed on the screen; and

FIG. 17 is a conceptual diagram showing an example of a series of pieces of processing in which a processor included in a computer issues a processing execution request to an external device via a network, the external device executes processing in accordance with the processing execution request, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings.

First, the terms used in the following description will be described.

CPU is an abbreviation for “central processing unit”. GPU is an abbreviation for “graphics processing unit”. GPGPU is an abbreviation for “general-purpose computing on graphics processing units”. APU is an abbreviation for “accelerated processing unit”. TPU is an abbreviation for “tensor processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. AI is an abbreviation for “artificial intelligence”. BLI is an abbreviation for “blue light imaging”. LCI is an abbreviation for “linked color imaging”. I/F is an abbreviation for “interface”. LAN is an abbreviation for “local area network”. WAN is an abbreviation for “wide area network”. 5G is an abbreviation for “5th generation mobile communication system”. IC is an abbreviation for “integrated circuit”.

In the following description, a processor with a reference numeral (hereinafter, simply referred to as “processor”) may be one physical or virtual operation device or a combination of a plurality of physical or virtual operation devices. Further, the processor may be one type of operation device or a combination of a plurality of types of operation devices. Examples of the operation device include a CPU, a GPU, a GPGPU, an APU, and a TPU.

In the following description, a memory with a reference numeral is a memory such as a RAM that temporarily stores information, and is used as a work memory by the processor.

In the following description, a storage with a reference numeral is one or a plurality of non-volatile storage devices storing various programs and various parameters or the like. Examples of the non-volatile storage device include a flash memory, a magnetic disk, or a magnetic tape. Other examples of the storage include a cloud storage.

In the following embodiment, an external I/F with a reference numeral controls the transmission and the reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), or Bluetooth (registered trademark).

In the following embodiment, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

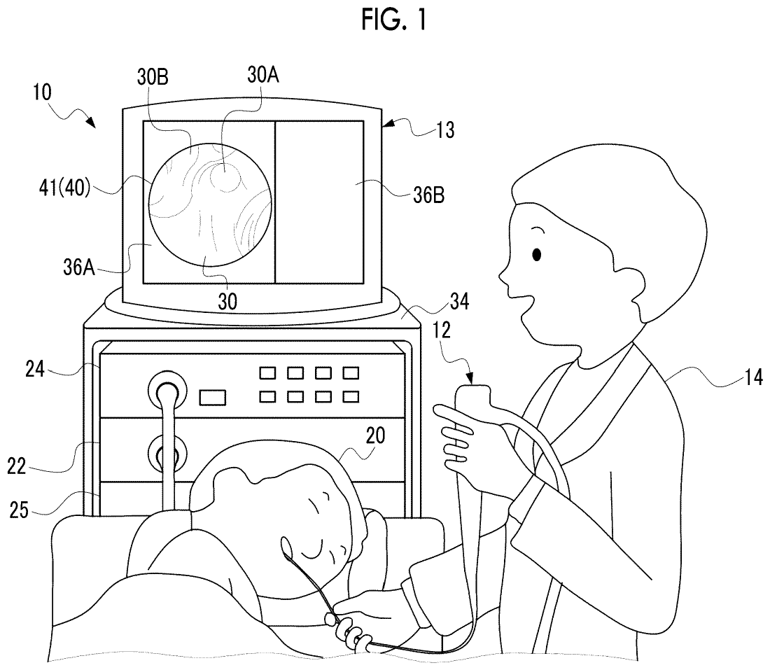

For example, as shown in FIG. 1, a duodenoscope system 10 comprises a duodenoscope 12 and a display device 13. The duodenoscope 12 is used by a doctor 14 in endoscopy. In the present embodiment, the duodenoscope system 10 is an example of an “endoscope system” according to the present disclosure, and the duodenoscope 12 is an example of an “endoscope” according to the present disclosure.

The duodenoscope system 10 is connected communicably to a communication device (not shown), and information obtained by the duodenoscope system 10 is transmitted to the communication device. The communication device receives the information transmitted from the duodenoscope system 10 and executes processing using the received information (for example, processing of recording the information in an electronic medical record).

The duodenoscope 12 is inserted into an upper gastrointestinal tract of a subject 20 (for example, a patient). The duodenoscope 12 is an endoscope having an optical imaging function of irradiating the inside of the upper gastrointestinal tract of the subject 20 with light and imaging reflected light obtained by being reflected by an intestinal wall 30 which is a part of the upper gastrointestinal tract of the subject 20. The duodenoscope 12 images the intestinal wall 30 to acquire an image showing an aspect of the intestinal wall 30, and outputs the image to the display device 13. The intestinal wall 30 imaged by the duodenoscope 12 is observed by the doctor 14 through the display device 13.

The duodenoscope 12 comprises a light source device 22, a control device 24, and a medical support device 25. The light source device 22, the control device 24, and the medical support device 25 are installed in a wagon 34. The wagon 34 is provided with a plurality of stages along an up-down direction, and the medical support device 25, the light source device 22, and the control device 24 are installed from a lower stage to an upper stage. The display device 13 is installed on the uppermost stage in the wagon 34.

The control device 24 controls the entire duodenoscope 12. The medical support device 25 executes various types of image processing on the image obtained by imaging the intestinal wall 30 via the duodenoscope 12 under the control of the control device 24.

The display device 13 displays various types of information including the image. Examples of the display device 13 include a liquid-crystal display and an EL display. In addition, a tablet terminal with a display may be used instead of or together with the display device 13.

The display device 13 displays plurality of screens side by side. In the example shown in FIG. 1, screens 36A and 36B are shown as examples of the plurality of screens. On the screen 36A, an intestinal wall image 40 obtained by imaging the intestinal wall 30 via the duodenoscope 12 is displayed. The intestinal wall image 40 shows the intestinal wall 30. Further, in the example shown in FIG. 1, the intestinal wall 30 includes a duodenal papilla 30A and a peripheral region 30B of the duodenal papilla 30A.

In the present embodiment, the intestinal wall image 40 is an example of an “intestinal wall image” according to the present disclosure. Further, in the present embodiment, the duodenal papilla 30A is an example of a “duodenal papilla” according to the present disclosure. Further, in the present embodiment, the peripheral region 30B is an example of a “peripheral region of the duodenal papilla” according to the present disclosure.

The intestinal wall image 40 is a moving image and is composed of a plurality of frames 41 in time series. The plurality of frames 41 in time series are displayed on the screen 36A at a predetermined frame rate (for example, several tens of frames/second).

The screen 36A is a main screen, whereas the screen 36B is a sub-screen. Various types of information for supporting a procedure using the duodenoscope 12 performed by the doctor 14 are displayed on the screen 36B. A size of the screen 36A and a size of the screen 36B may be fixed, or may be changed in response to an instruction issued from the doctor 14 to the duodenoscope system 10 or in accordance with various conditions.

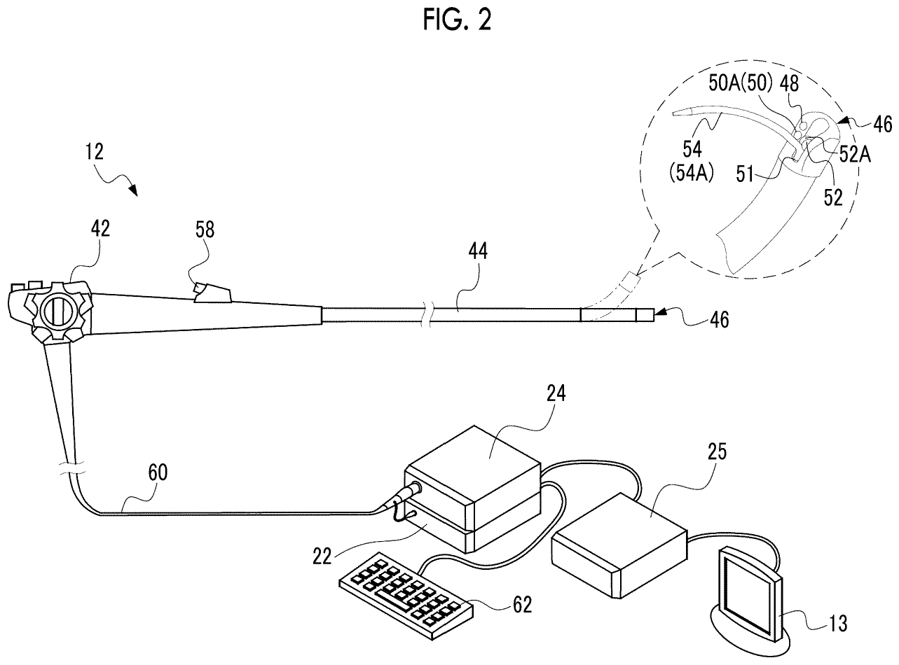

As shown in FIG. 2 as an example, the duodenoscope 12 comprises an operating part 42 and an insertion part 44. The insertion part 44 is partially bent by operating the operating part 42. The insertion part 44 is inserted while being bent in accordance with a shape of the upper gastrointestinal tract (for example, a shape of a duodenum) in accordance with an operation of the operating part 42 performed by the doctor 14.

A camera 48, an illumination device 50, a treatment opening 51, and an elevating mechanism 52 are provided at a distal end part 46 of the insertion part 44. The camera 48 and the illumination device 50 are provided on a side surface of the distal end part 46. That is, the duodenoscope 12 is configured as a side-view endoscope, and an interior wall of the duodenum, that is, the intestinal wall 30, is easily observed.

The camera 48 is a device that generates the intestinal wall image 40 as a medical image by imaging an inside of a body of the subject 20, that is, the inside of the upper gastrointestinal tract. Examples of the camera 48 include a CMOS camera. However, this is merely an example, and another type of camera such as a CCD camera may be used.

The illumination device 50 includes an illumination window 50A. The illumination device 50 emits light through the illumination window 50A. Examples of the type of the light emitted from the illumination device 50 include visible light (for example, white light) and invisible light (for example, near-infrared light). In addition, the illumination device 50 emits special light through the illumination window 50A. Examples of the special light include light for BLI and/or light for LCI. The camera 48 images the inside of the subject 20 by using an optical method in a state in which the inside of the subject 20 is irradiated with the light by the illumination device 50.

The treatment opening 51 is used as a treatment tool protruding port through which a treatment tool 54 protrudes from the distal end part 46, a suction port for suctioning blood, internal waste, and the like, and a delivery port for delivering a fluid.

The treatment tool 54 protrudes from the treatment opening 51 in accordance with the operation of the doctor 14. The treatment tool 54 is inserted into the insertion part 44 from a treatment tool insertion port 58. The treatment tool 54 passes through the inside of the insertion part 44 through the treatment tool insertion port 58 and protrudes from the treatment opening 51 into the body of the subject 20. In the example shown in FIG. 2, a cannula 54A protrudes from the treatment opening 51 as the treatment tool 54. The cannula 54A is merely an example of the treatment tool 54, and other examples of the treatment tool 54 include a catheter, a guide wire, a papillotomy knife, and a snare.

The elevating mechanism 52 changes a protrusion direction of the treatment tool 54 protruding from the treatment opening 51. The elevating mechanism 52 comprises a guide 52A, and the guide 52A rises with respect to the protrusion direction of the treatment tool 54, so that the protrusion direction of the treatment tool 54 is changed along the guide 52A. Therefore, it is easy to cause the treatment tool 54 to protrude toward the intestinal wall 30. In the example shown in FIG. 2, the protrusion direction of the treatment tool 54 is changed to a direction orthogonal to a traveling direction of the distal end part 46 by the elevating mechanism 52. The elevating mechanism 52 is operated by the doctor 14 using the operating part 42. As a result, a degree of change in the protrusion direction of the treatment tool 54 is adjusted.

The duodenoscope 12 is connected to the light source device 22 and the control device 24 via a universal cord 60. A reception device 62 is connected to the control device 24. In addition, the medical support device 25 is connected to the control device 24. Further, the display device 13 is connected to the medical support device 25. That is, the control device 24 is connected to the display device 13 via the medical support device 25.

It should be noted that, here, since the medical support device 25 is described as an external device for expanding the functions executed by the control device 24, the form example is described in which the control device 24 and the display device 13 are indirectly connected to each other via the medical support device 25, but this is merely an example. For example, the display device 13 may be directly connected to the control device 24. In this case, for example, the functions of the medical support device 25 need only be provided in the control device 24, or a function of executing, via a server (not shown), the same process as a process (for example, a medical support process described below) executed by the medical support device 25 and receiving and using a processing result from the server need only be provided in the control device 24.

The reception device 62 receives an instruction from a user (for example, the doctor 14) and outputs the received instruction to the control device 24 as an electric signal. Examples of the reception device 62 include a keyboard, a mouse, a touch panel, a foot switch, and a microphone.

The control device 24 controls the light source device 22, controls the transmission and the reception of various signals with the camera 48, and controls the transmission and the reception of various signals with the medical support device 25.

The light source device 22 emits light under the control of the control device 24 to supply the light to the illumination device 50. The illumination device 50 is provided with a built-in light guide, and the light supplied from the light source device 22 is emitted from the illumination window 50A via the light guide. The control device 24 causes the camera 48 to execute the imaging, acquires the intestinal wall image 40 (see FIG. 1) from the camera 48, and outputs the intestinal wall image 40 to a predetermined output destination (for example, the medical support device 25).

The medical support device 25 executes various types of image processing on the intestinal wall image 40 input from the control device 24. The medical support device 25 outputs the intestinal wall image 40 on which various types of image processing are executed, to a predetermined output destination (for example, the display device 13).

It should be noted that, here, the form example is described in which the intestinal wall image 40 output from the control device 24 is output to the display device 13 via the medical support device 25, but this is merely an example. The control device 24 and the display device 13 may be connected to each other, and the intestinal wall image 40 on which the image processing has been performed by the medical support device 25 may be displayed on the display device 13 via the control device 24.

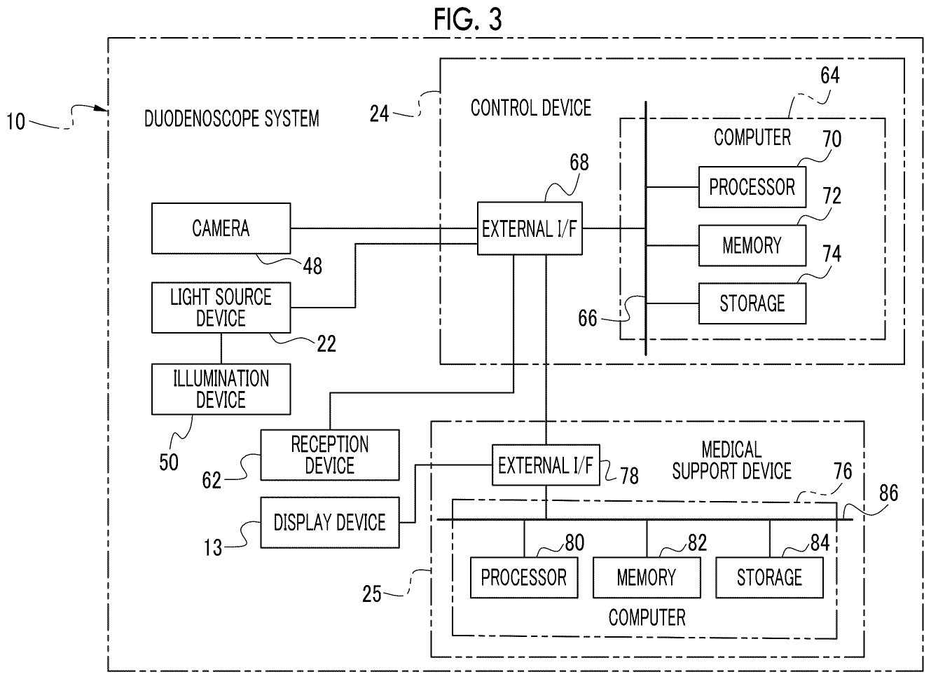

As shown in FIG. 3 as an example, the control device 24 comprises a computer 64, a bus 66, and an external I/F 68. The computer 64 comprises a processor 70, a memory 72, and a storage 74. The processor 70, the memory 72, the storage 74, and the external I/F 68 are connected to a bus 66.

The external I/F 68 controls the transmission and the reception of various types of information between one or more devices (hereinafter also referred to as “first external devices”) present outside the control device 24 and the processor 70.

The camera 48 is connected to the external I/F 68 as one of the first external devices, and the external I/F 68 controls the transmission and the reception of various types of information between the camera 48 and the processor 70. The processor 70 controls the camera 48 via the external I/F 68. In addition, the processor 70 acquires, via the external I/F 68, the intestinal wall image 40 (see FIG. 1) obtained by imaging the inside of the body of the subject 20 via the camera 48.

The light source device 22 is connected to the external I/F 68 as one of the first external devices, and the external I/F 68 controls the transmission and the reception of various types of information between the light source device 22 and the processor 70. The light source device 22 supplies the light to the illumination device 50 under the control of the processor 70. The illumination device 50 emits the light supplied from the light source device 22.

The reception device 62 is connected to the external I/F 68 as one of the first external devices, and the processor 70 acquires the instruction received by the reception device 62 via the external I/F 68 and executes processing in response to the acquired instruction.

The medical support device 25 comprises a computer 76 and an external I/F 78. The computer 76 comprises a processor 80, a memory 82, and a storage 84. The processor 80, the memory 82, the storage 84, and the external I/F 78 are connected to a bus 86. In the present embodiment, the medical support device 25 is an example of a “medical support device” according to the present disclosure, the computer 76 is an example of a “computer” according to the present disclosure, and the processor 80 is an example of a “processor” according to the present disclosure.

The external I/F 78 controls the transmission and the reception of various types of information between one or more devices (hereinafter also referred to as “second external devices”) present outside the medical support device 25 and the processor 80.

The control device 24 is connected to the external I/F 78 as one of the second external devices. In the example shown in FIG. 3, the external I/F 68 of the control device 24 is connected to the external I/F 78. The external I/F 78 controls the transmission and the reception of various types of information between the processor 80 of the medical support device 25 and the processor 70 of the control device 24. For example, the processor 80 acquires the intestinal wall image 40 (see FIG. 1) from the processor 70 of the control device 24 via the external I/Fs 68 and 78, and executes various types of image processing on the acquired intestinal wall image 40.

The display device 13 is connected to the external I/F 78 as one of the second external devices. The processor 80 controls the display device 13 via the external I/F 78 to display various types of information (for example, the intestinal wall image 40 on which various types of image processing have been executed) on the display device 13.

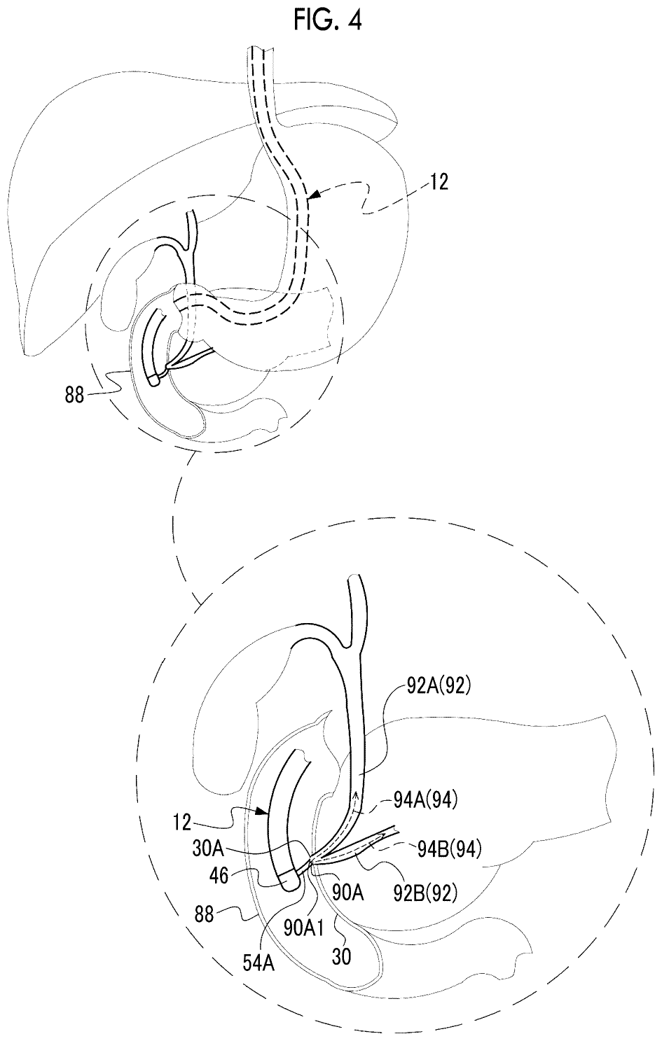

As one of medical treatments for the duodenum using the duodenoscope 12, a treatment called endoscopic retrograde cholangiopancreatography (ERCP) examination is known. As shown in FIG. 4 as an example, in the ERCP examination, for example, first, the duodenoscope 12 is inserted into a duodenum 88 through an esophagus and a stomach. In this case, an insertion state of the duodenoscope 12 may be checked by using an X-ray image obtained by X-ray imaging. Then, the distal end part 46 of the duodenoscope 12 reaches the vicinity of the duodenal papilla 30A present in the intestinal wall 30.

In the ERCP examination, for example, the cannula 54A is inserted from a lumen side of the duodenum 88 into the duodenal papilla 30A. Here, the duodenal papilla 30A is a part that is elevated from the intestinal wall 30. In a papillary elevation 90A, which is a distal end part of the duodenal papilla 30A, an opening part 90A1 is present, which leads to an end part of one or more tubes 92 leading to an internal organ (for example, a gallbladder and a pancreas), that is, which leads to the tube 92. In other words, the tube 92 communicates with the opening part 90A1 present in the papillary elevation 90A.

Examples of one or more tubes 92 include a bile duct 92A and a pancreatic duct 92B. The opening part 90A1 may be individually present with respect to each of the bile duct 92A and the pancreatic duct 92B, or may be present in common to the bile duct 92A and the pancreatic duct 92B.

In the ERCP examination, the X-ray imaging is executed in a state in which a contrast agent is injected into the tube 92 through the opening part 90A1. Here, in a case in which the doctor 14 inserts the cannula 54A into the tube 92, it is necessary to accurately understand a running direction 94 of the tube 92. In particular, since the running direction 94 in the vicinity of the opening part 90A1 substantially matches an insertion direction of the cannula 54A with respect to the opening part 90A1, it is very important for the doctor 14 to visually understand the running direction 94 in the vicinity of the opening part 90A1.

Examples of the running direction 94 of the tube 92 include a running direction 94A of the bile duct 92A and a running direction 94B of the pancreatic duct 92B. It is effective for the doctor 14 to visually understand the running direction 94A in a case in which the cannula 54A is inserted into the bile duct 92A, and it is effective for the doctor 14 to visually understand the running direction 94B in a case in which the cannula 54A is inserted into the pancreatic duct 92B.

A difficulty level of the insertion of the cannula 54A into the tube 92, that is, a difficulty level of intubation into the duodenal papilla 30A, varies greatly depending on the conditions. For example, depending on a combination of a confluence form of the bile duct 92A and the pancreatic duct 92B (hereinafter, simply referred to as a “confluence form”) in the duodenal papilla 30A and a type of the opening part 90A1, the intubation into the duodenal papilla 30A is more difficult. In addition, the intubation into the duodenal papilla 30A is made more difficult due to a shape of the duodenal papilla 30A, a size of the duodenal papilla 30A, a length of an oral side elevation of the duodenal papilla 30A, an aspect of the peripheral region 30B (for example, whether or not there is a diverticulum), and/or deformation of the duodenum.

Therefore, in a case in which the difficulty level of the intubation into the duodenal papilla 30A is equal to or higher than a certain difficulty level (for example, a difficulty level in which a doctor with an average ability to perform the intubation into the duodenal papilla wants information that is useful for supporting the intubation into the duodenal papilla), it is preferable that support information, which is the information that is useful for supporting the intubation into the duodenal papilla 30A, is displayed on the screen 36A and/or 36B.

However, the doctor 14 during the procedure does not have time to determine by himself/herself whether or not the difficulty level of the intubation into the duodenal papilla 30A is equal to or higher than a certain difficulty level and to execute an operation for displaying the necessary support information on the screen 36A and/or 36B.

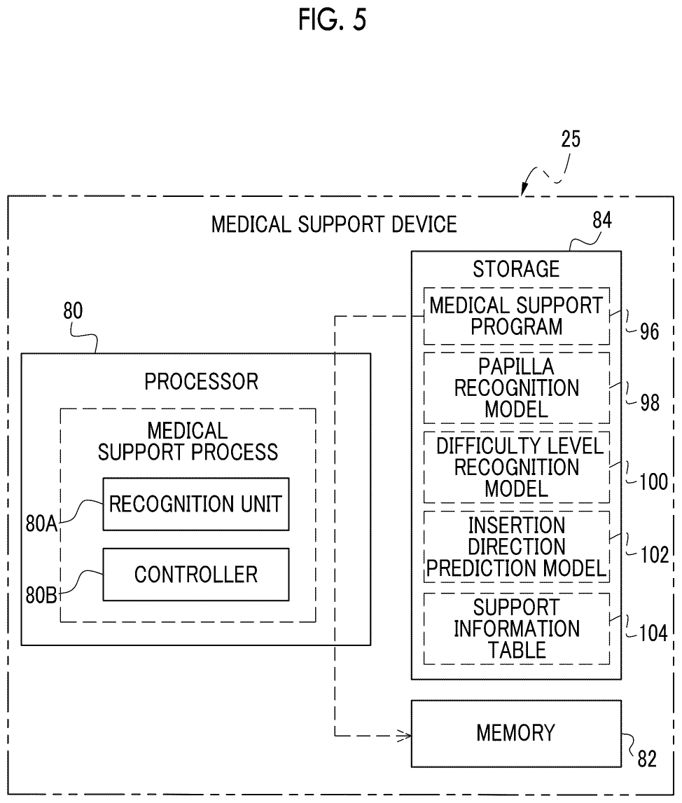

Therefore, in view of such circumstances, in the present embodiment, as shown in FIG. 5 as an example, the medical support process is executed by the processor 80 of the medical support device 25. A medical support program 96 is stored in the storage 84. In the present embodiment, the medical support program 96 is an example of a “program” according to the present disclosure. The processor 80 reads out the medical support program 96 from the storage 84 and executes the readout medical support program 96 on the memory 82 to execute the medical support process. The medical support process is implemented by the processor 80 operating as a recognition unit 80A and a controller 80B in accordance with the medical support program 96 executed on the memory 82.

The storage 84 stores a papilla recognition model 98, a difficulty level recognition model 100, an insertion direction prediction model 102, and a support information table 104. As will be described below, the difficulty level recognition model 100 and the insertion direction prediction model 102 are used by the recognition unit 80A, and the support information table 104 is used by the controller 80B.

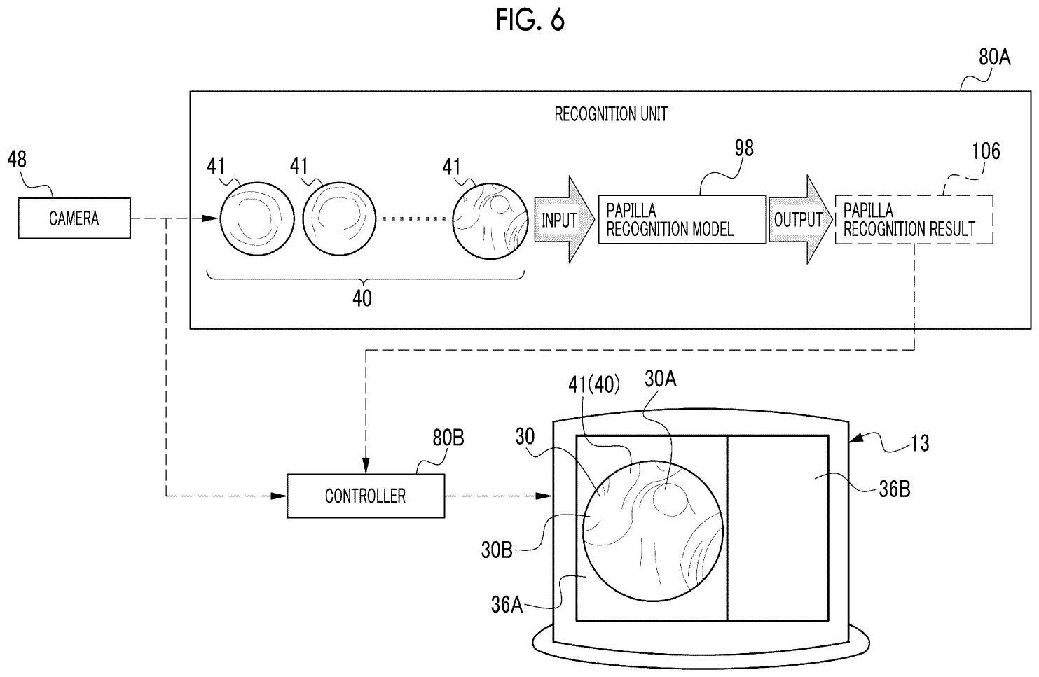

As shown in FIG. 6 as an example, the recognition unit 80A and the controller 80B acquire the intestinal wall image 40. For example, the recognition unit 80A and the controller 80B acquire the intestinal wall image 40 generated by being captured by the camera 48 in accordance with an imaging frame rate (for example, several tens of frames/second) from the camera 48 in units of one frame. The controller 80B displays, on the screen 36A, the intestinal wall image 40 acquired from the camera 48.

Meanwhile, the recognition unit 80A recognizes the duodenal papilla 30A and the peripheral region 30B shown in the frame 41 based on each of the plurality of frames 41 included in the intestinal wall image 40 acquired from the camera 48. In the present embodiment, in order to implement this, the recognition unit 80A recognizes the duodenal papilla 30A and the peripheral region 30B by using an AI method. Here, the recognition processing using the papilla recognition model 98 is executed. In the present embodiment, the papilla recognition model 98 is an example of a “second trained model” according to the present disclosure.

The papilla recognition model 98 is a trained model for object recognition using a segmentation method via AI, and is obtained by executing machine learning for the frame 41. The papilla recognition model 98 has been optimized by training a neural network through machine learning using first training data that is a data set including a plurality of data (that is, data for a plurality of frames) in which first example data and first correct answer data are associated with each other. That is, the papilla recognition model 98 is a trained model that has been optimized to receive input of first example data to generate first correct answer data.

The first example data is an image that corresponds to the frame 41 (in other words, a sample image assuming the frame 41). A first example of the image that corresponds to the frame 41 is an optical image actually obtained by a duodenoscope having the same configuration as the duodenoscope 12. A second example of the image that corresponds to the frame 41 is a virtually generated image (for example, an image generated by generative AI).

The first correct answer data refers to correct answer data (that is, an annotation) for the first example data. That is, the first correct answer data is information for specifying the duodenal papilla and the peripheral region of the duodenal papilla, which are shown in the image used as the first example data, in a distinguishable manner. Here, as an example of the first correct answer data, annotations are used for specifying geometrical characteristics (for example, the position, the size, and the shape) of the duodenal papilla shown in the image used as the first example data, anatomical features (for example, the type of the opening part and the length of the oral side elevation) of the duodenal papilla, geometrical characteristics (for example, the position, the size, and the shape) of the peripheral region of the duodenal papilla, and anatomical features (for example, the presence or absence of the diverticulum) of the peripheral region of the duodenal papilla.

The recognition unit 80A inputs the frame 41 to the papilla recognition model 98 at a frame rate at which the frame 41 is displayed on the screen 36A. The papilla recognition model 98 recognizes the duodenal papilla 30A and the peripheral region 30B shown in the input frame 41 each time the frame 41 is input, generates a papilla recognition result 106, which is a recognition result, and outputs the generated papilla recognition result 106 to the controller 80B. The papilla recognition result 106 includes information indicating each of the geometrical characteristics of the duodenal papilla 30A, the anatomical features (for example, the type of the opening part 90A1 and the length of the oral side elevation) of the duodenal papilla 30A, the geometrical characteristics of the peripheral region 30B, and the anatomical features (for example, the presence or absence of the diverticulum) of the peripheral region 30B.

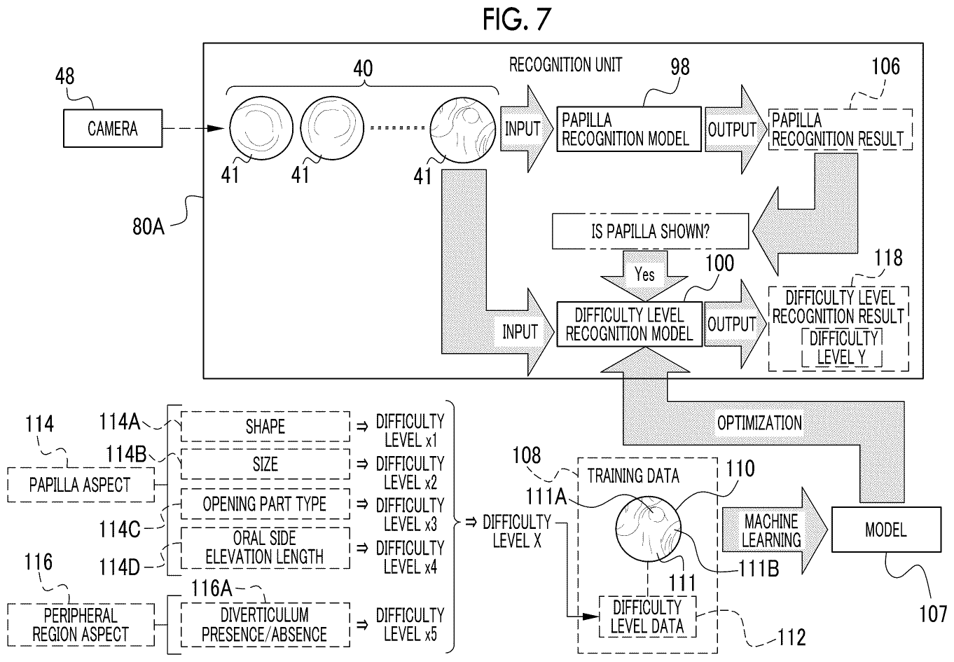

As shown in FIG. 7 as an example, the recognition unit 80A determines whether or not the duodenal papilla 30A and the peripheral region 30B are shown in the frame 41 input to the papilla recognition model 98 to obtain the papilla recognition result 106, based on the papilla recognition result 106. Here, in a case in which the duodenal papilla 30A and the peripheral region 30B are shown in the frame 41 input to the papilla recognition model 98 to obtain the papilla recognition result 106, the recognition unit 80A inputs the frame 41 input to the papilla recognition model 98 to obtain the papilla recognition result 106, to the difficulty level recognition model 100.

The difficulty level recognition model 100 is a trained model for image processing via AI, and is obtained by executing the machine learning for the frame 41. The difficulty level recognition model 100 has been optimized by training a neural network through machine learning using second training data that is a data set including a plurality of data (that is, data for a plurality of frames) in which second example data and second correct answer data are associated with each other. That is, the difficulty level recognition model 100 is a trained model that has been optimized to receive input of second example data to generate second correct answer data.

In the example shown in FIG. 7, training data 108 is shown as the second training data. The training data 108 includes a sample image 110 as an example of the second example data and difficulty level data 112 as an example of the second correct answer data, and the sample image 110 and the difficulty level data 112 are associated with each other.

The sample image 110 is the same image as the image used in the above-described first example data. An intestinal wall 111 of the duodenum, a duodenal papilla 111A, and a peripheral region 111B of the duodenal papilla 111A are shown in the sample image 110.

The difficulty level data 112 is data indicating the difficulty level of the intubation into the duodenal papilla 111A. The difficulty level of the intubation into the duodenal papilla 111A is determined based on the duodenal papilla 111A and the peripheral region 111B. In the example shown in FIG. 7, a difficulty level X is shown as the difficulty level of the intubation into the duodenal papilla 111A. The difficulty level data 112 is data indicating the difficulty level X.

In the example shown in FIG. 7, the difficulty level X is determined based on a papilla aspect 114 and a peripheral region aspect 116. The papilla aspect 114 is an aspect of the duodenal papilla 111A. The papilla aspect 114 includes a shape 114A of the duodenal papilla 111A, a size 114B of the duodenal papilla 111A, an opening part type 114C (that is, a type of an opening part of the duodenal papilla 111A), and an oral side elevation length 114D (that is, a length of an oral side elevation of the duodenal papilla 111A).

Meanwhile, the peripheral region aspect 116 is an aspect of the peripheral region 111B. The peripheral region aspect 116 includes diverticulum presence/absence 116A (that is, the presence or absence of the diverticulum).

A difficulty level x1 is given to a shape 114A. A difficulty level x2 is given to a size 114B. A difficulty level x3 is given to an opening part type 114C. A difficulty level x4 is given to an oral side elevation length 114D. A difficulty level x5 is given to diverticulum presence/absence 116A.

The difficulty level x1 means a difficulty level of the intubation into the duodenal papilla 111A in a case of the shape 114A. The difficulty level x2 means a difficulty level of the intubation into the duodenal papilla 111A in a case of the size 114B. The difficulty level x3 means a difficulty level of the intubation into the duodenal papilla 111A in a case of the opening part type 114C. The difficulty level x4 means a difficulty level of the intubation into the duodenal papilla 111A in a case of the oral side elevation length 114D. The difficulty level x5 means a difficulty level of the intubation into the duodenal papilla 111A in a case of the diverticulum presence/absence 116A.

For example, the difficulty level X is a difficulty level that is comprehensively determined from the difficulty levels x1 to x5 by an annotator (that is, a creator of the difficulty level data 112). An example of the difficulty level that is comprehensively determined from the difficulty levels x1 to x5 is an average difficulty level of the difficulty levels x1 to x5.

The difficulty level recognition model 100 is a trained model in which a model 107 (for example, a neural network) has been optimized by executing machine learning using the training data 108 on the model 107.

The recognition unit 80A inputs the frame 41 to the difficulty level recognition model 100 at the frame rate at which the frame 41 is displayed on the screen 36A. The difficulty level recognition model 100 recognizes the difficulty level of the intubation into the duodenal papilla 30A shown in the input frame 41 each time the frame 41 is input, and generates a difficulty level recognition result 118 which is the recognition result. The difficulty level recognition result 118 is output to the controller 80B.

The difficulty level recognition result 118 includes a difficulty level Y. The difficulty level Y means a difficulty level of the intubation into the duodenal papilla 30A shown in the frame 41 input to the difficulty level recognition model 100.

The difficulty level recognition model 100 is a trained model obtained through the machine learning using the training data 108 including the difficulty level data 112 determined based on the duodenal papilla 111A and the peripheral region 111B. Therefore, the difficulty level Y included in the difficulty level recognition result 118 generated by the difficulty level recognition model 100 can be said to be a difficulty level determined based on the duodenal papilla 30A and the peripheral region 30B.

In addition, the difficulty level recognition model 100 is a trained model obtained through the machine learning using the training data 108 including the difficulty level data 112 determined based on the papilla aspect 114 and the peripheral region aspect 116. Therefore, the difficulty level Y included in the difficulty level recognition result 118 generated by the difficulty level recognition model 100 can be said to be a difficulty level determined based on the aspect of the duodenal papilla 30A and the aspect of the peripheral region 30B.

In addition, the difficulty level recognition model 100 is a trained model obtained through the machine learning using the training data 108 including the difficulty level data 112 determined based on the shape 114A, the size 114B, the opening part type 114C, the oral side elevation length 114D, and the diverticulum presence/absence 116A. Therefore, the difficulty level Y included in the difficulty level recognition result 118 generated by the difficulty level recognition model 100 can be said to be a difficulty level determined based on the shape of the duodenal papilla 30A, the size of the duodenal papilla 30A, the type of the opening part 90A1 of the duodenal papilla 30A, the length of the oral side elevation of the duodenal papilla 30A, and the presence or absence of the diverticulum in the peripheral region 30B.

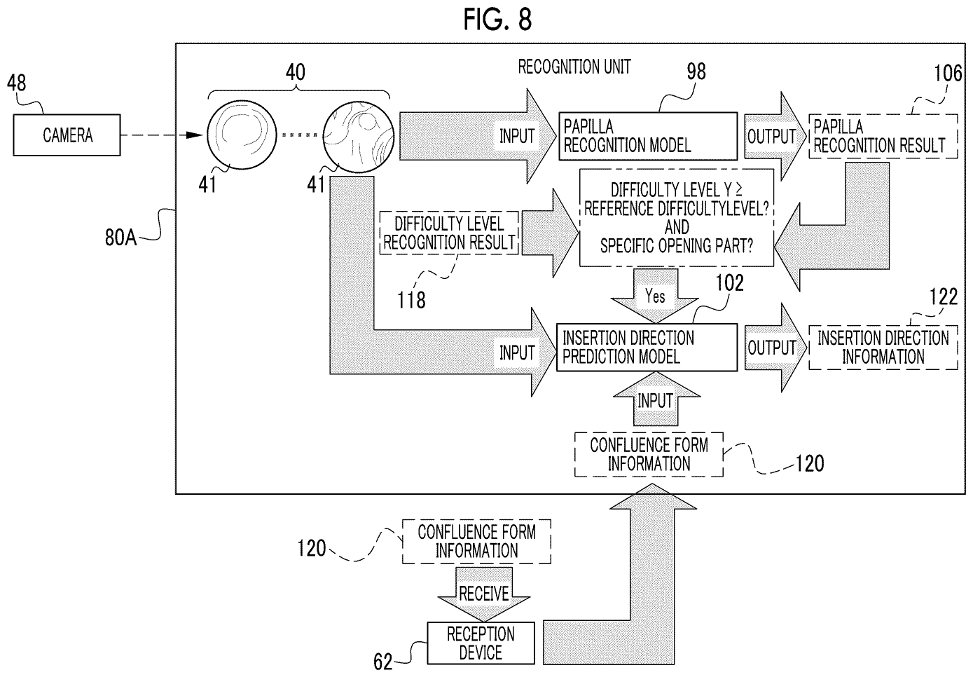

As shown in FIG. 8 as an example, the recognition unit 80A determines whether or not a specific opening part is shown in the frame 41 input to the papilla recognition model 98 to obtain the papilla recognition result 106, based on the papilla recognition result 106. Here, the specific opening part means the opening part 90A1 (for example, the opening part 90A1 of a nodular type or a villous type) for which it is generally recognized that it is difficult to perform the intubation into the duodenal papilla 30A. In addition, the recognition unit 80A determines whether or not the difficulty level Y included in the difficulty level recognition result 118 is equal to or higher than a reference difficulty level.

Here, a first example of the reference difficulty level is a difficulty level in which a time required for a doctor, who has an ability lower than the average ability to perform the intubation into the duodenal papilla, from the start of the intubation to the end of the intubation is expected to be equal to or longer than a certain time (for example, 10 minutes). A second example of the reference difficulty level is a difficulty level in which a time required for a doctor, who has the average ability to perform the intubation into the duodenal papilla, from the start of the intubation to the end of the intubation is expected to be equal to or longer than a certain time (for example, 5 minutes). In addition, a third example of the reference difficulty level is a difficulty level in which a time required for a doctor, who has an ability higher than the average ability to perform the intubation into the duodenal papilla, from the start of the intubation to the end of the intubation is expected to be equal to or longer than a certain time (for example, 2 minutes). It should be noted that the reference difficulty level is statistically derived in advance based on a plurality of cases in which the intubation into the duodenal papilla is actually executed by each of a plurality of doctors.

In a case in which the specific opening part is shown in the frame 41 input to the papilla recognition model 98 to obtain the papilla recognition result 106 and the difficulty level Y included in the difficulty level recognition result 118 is equal to or higher than the reference difficulty level, the recognition unit 80A predicts a direction in which the cannula 54A is inserted into the duodenal papilla 30A (hereinafter, simply referred to as an “insertion direction”), based on the insertion direction prediction model 102. The insertion direction includes a direction in which the cannula 54A is inserted into the bile duct 92A and a direction in which the cannula 54A is inserted into the pancreatic duct 92B.

The insertion direction prediction model 102 is a trained model for prediction via AI, and is obtained by executing the machine learning for the frame 41. The insertion direction prediction model 102 has been optimized by training a neural network through machine learning using third training data that is a data set including a plurality of data (that is, data for a plurality of frames) in which third example data and third correct answer data are associated with each other. That is, the insertion direction prediction model 102 is a trained model that has been optimized to receive input of third example data to generate third correct answer data.

The third example data includes the image used in the above-described first example data and the confluence form. The third correct answer data refers to correct answer data (that is, an annotation) for the third example data. That is, the third correct answer data is information for specifying the insertion direction (for example, the insertion direction applied to the bile duct 92A and the insertion direction applied to the pancreatic duct 92B) suitable for the duodenal papilla shown in the image used as the third example data.

The recognition unit 80A inputs the same frame 41 as the frame 41 input to the papilla recognition model 98 to obtain the papilla recognition result 106 at the frame rate at which the frame 41 is displayed on the screen 36A, and confluence form information 120 to the insertion direction prediction model 102. Here, the confluence form information 120 is received by the reception device 62. The confluence form information 120 is, for example, information indicating the confluence form that is specified in advance from an examination result obtained by performing a CT examination, an MRI examination, or the like on the subject 20.

In a case in which the frame 41 and the confluence form information 120 are input to the insertion direction prediction model 102, the insertion direction prediction model 102 generates insertion direction information 122 that is information for specifying the insertion direction, and outputs the generated insertion direction information 122. In the present embodiment, the insertion direction information 122 is an example of “support information” according to the present disclosure.

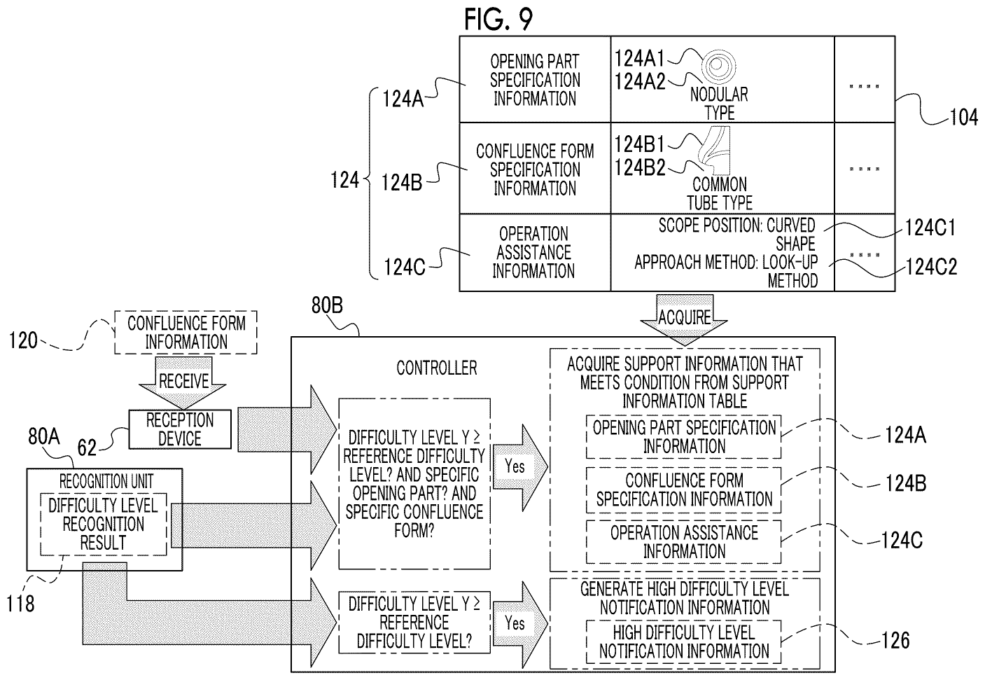

As shown in FIG. 9 as an example, the support information table 104 includes support information 124. The support information 124 includes a plurality of pieces of opening part specification information 124A, a plurality of pieces of confluence form specification information 124B, and a plurality of pieces of operation assistance information 124C. The opening part specification information 124A is information for specifying the opening part 90A1. The confluence form specification information 124B is information for specifying the confluence form. The operation assistance information 124C is information for assisting the operation of the duodenoscope 12. In the present embodiment, the operation assistance information 124C is an example of “operation assistance information” according to the present disclosure.

The support information table 104 is provided with the opening part specification information 124A for each type of the opening part 90A1. The opening part specification information 124A is information in which an opening part schema 124A1, which is a schema for specifying the type of the opening part 90A1, and opening part text 124A2, which is text for specifying the type of the opening part 90A1, are associated with each other.

The support information table 104 is provided with the confluence form specification information 124B for each confluence form. In addition, the confluence form specification information 124B is associated with the opening part specification information 124A for each type of the opening part 90A1. The confluence form specification information 124B is information in which a confluence form schema 124B1, which is a schema for specifying the confluence form, and confluence form text 124B2, which is text for specifying the confluence form, are associated with each other.

The operation assistance information 124C is associated with the opening part specification information 124A for each type of the opening part 90A1. In addition, the operation assistance information 124C is associated with the confluence form specification information 124B for each confluence form. The operation assistance information 124C is information in which scope position information 124C1 and approach method information 124C2 are associated with each other. The scope position information 124C1 is information (here, as an example, text) for specifying a position (for example, a position at which an objective lens of the camera 48 faces the opening part 90A1 in front view) recommended as a position of the duodenoscope 12. The approach method information 124C2 is information (here, as an example, text) for specifying a method of approach to the opening part 90A1 (for example, a proximity method, a look-up method, and the like).

In a case in which the difficulty level Y included in the difficulty level recognition result 118 acquired from the recognition unit 80A is equal to or higher than the reference difficulty level, the controller 80B generates high difficulty level notification information 126. The high difficulty level notification information 126 is information for giving notification that the difficulty level Y is equal to or higher than the reference difficulty level. An example of the high difficulty level notification information 126 includes text indicating that the difficulty level Y is equal to or higher than the reference difficulty level. In the present embodiment, the high difficulty level notification information 126 is an example of “information indicating that the difficulty level is equal to or higher than the reference difficulty level” according to the present disclosure. In addition, in the present embodiment, the high difficulty level notification information 126 is an example of “support information” according to the present disclosure.

In a case in which the difficulty level Y included in the difficulty level recognition result 118 acquired from the recognition unit 80A is equal to or higher than the reference difficulty level, the specific opening part is included in the frame 41 input to the papilla recognition model 98 to obtain the papilla recognition result 106, and the confluence form specified by the confluence form information 120 received by the reception device 62 is a specific confluence form (for example, a partition wall type or a common tube type), the controller 80B acquires the support information 124 that meets the conditions from the support information table 104. Here, the support information 124 that meets the condition means, for example, the opening part specification information 124A corresponding to the specific opening part, the confluence form specification information 124B corresponding to the specific confluence form, and the operation assistance information 124C corresponding to the opening part specification information 124A and the confluence form specification information 124B.

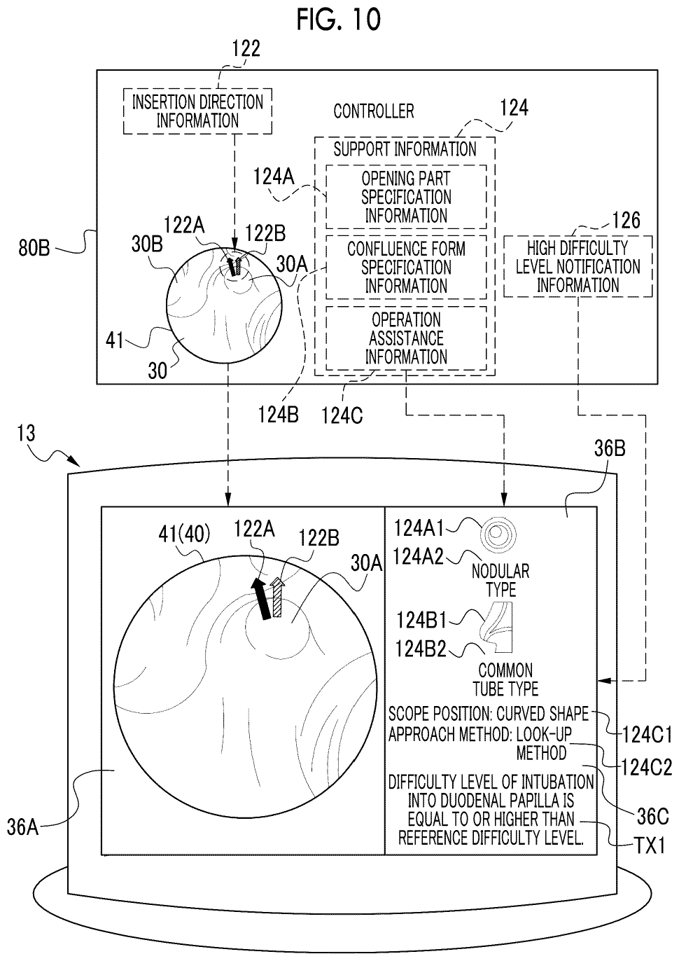

As shown in FIG. 10 as an example, the controller 80B outputs the insertion direction information 122 obtained from the insertion direction prediction model 102, the support information 124 acquired from the support information table 104, and the high difficulty level notification information 126 to the display device 13.

That is, in a case in which the difficulty level Y included in the difficulty level recognition result 118 is equal to or higher than the reference difficulty level and the specific opening part is included in the frame 41 input to the papilla recognition model 98, the insertion direction information 122 is output to the display device 13. In addition, in a case in which the difficulty level Y included in the difficulty level recognition result 118 is equal to or higher than the reference difficulty level, the specific opening part is included in the frame 41 input to the papilla recognition model 98, and the confluence form specified from the confluence form information 120 received by the reception device 62 is the specific confluence form, the support information 124 is output to the display device 13. Further, in a case in which the difficulty level Y included in the difficulty level recognition result 118 is equal to or higher than the reference difficulty level, the high difficulty level notification information 126 is output to the display device 13.

As described above, the insertion direction information 122, the support information 124, and the high difficulty level notification information 126 are output to the display device 13, and thus visible information is displayed on the screens 36A and 36B of the display device 13. For example, the controller 80B converts the insertion direction information 122 into an image, and displays the converted image on the screen 36A. In the example shown in FIG. 10, arrow marks 122A and 122B, which are obtained by converting the insertion direction information 122 into an image, are displayed in a superimposed manner on the frame 41 displayed on the screen 36A. A direction indicated by the arrow mark 122A is the direction in which the cannula 54A is inserted into the bile duct 92A, and a direction indicated by the arrow mark 122B is the direction in which the cannula 54A is inserted into the pancreatic duct 92B. Further, the controller 80B displays the support information 124 on the screen 36B. Further, the controller 80B displays the high difficulty level notification information 126 on the screen 36B as text TX1.

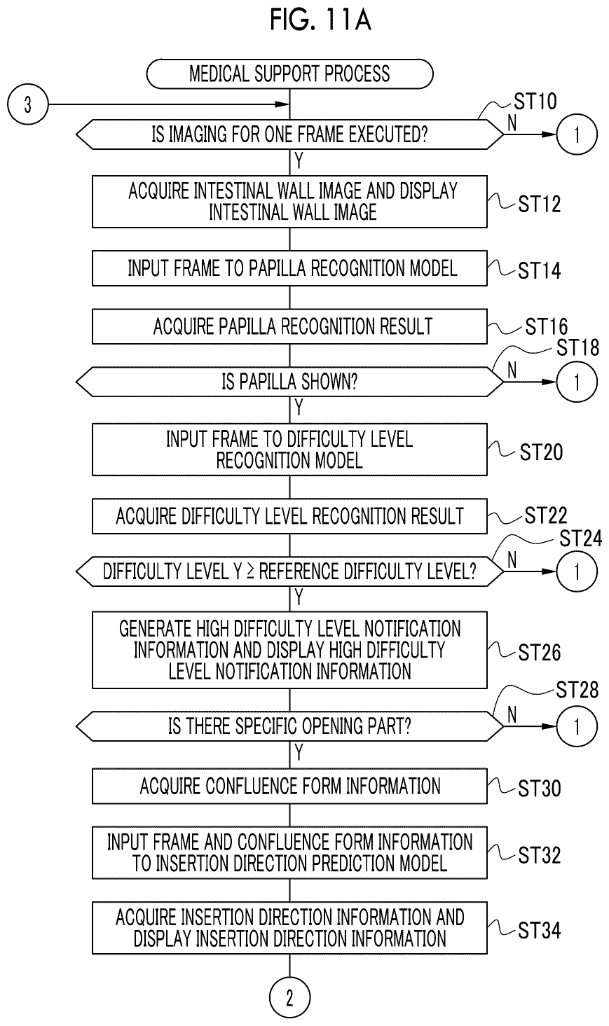

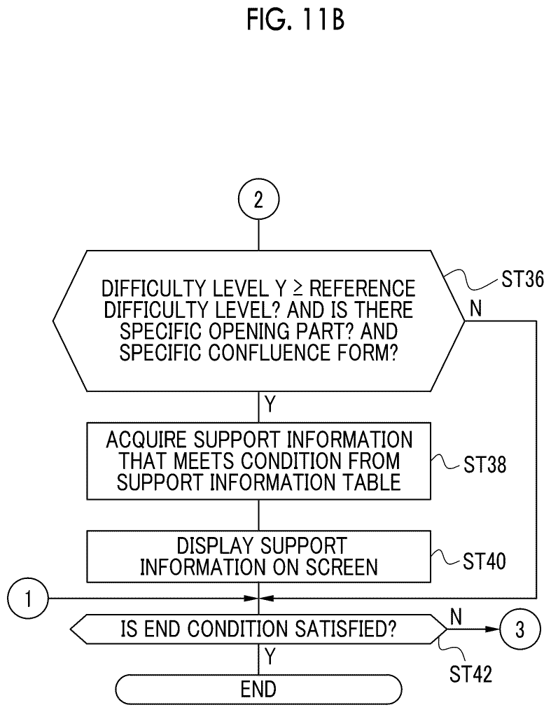

Next, an operation of a portion of the duodenoscope system 10 according to the present disclosure will be described with reference to FIGS. 11A and 11B.

FIGS. 11A and 11B show an example of a flow of the medical support process executed by the processor 80. The flow of the medical support process shown in FIGS. 11A and 11B is an example of a “medical support method” according to the present disclosure.

In the medical support process shown in FIG. 11A, first, in step ST10, the controller 80B determines whether or not the camera 48 images the intestinal wall 30 for one frame. In step ST10, in a case in which the camera 48 does not image the intestinal wall 30 for one frame, a negative determination is made, and the medical support process proceeds to step ST42 shown in FIG. 11B. In step ST10, in a case in which the camera 48 images the intestinal wall 30 for one frame, an affirmative determination is made, and the medical support process proceeds to step ST12.

In step ST12, the recognition unit 80A and the controller 80B acquire the intestinal wall image 40 from the camera 48. The controller 80B displays the intestinal wall image 40 on the screen 36A. After the processing in step ST12 is executed, the medical support process proceeds to step ST14.

In step ST14, the recognition unit 80A inputs the frame 41 included in the intestinal wall image 40 acquired in step ST12 to the papilla recognition model 98. As a result, the papilla recognition model 98 generates the papilla recognition result 106. After the processing in step ST14 is executed, the medical support process proceeds to step ST16.

In step ST16, the recognition unit 80A acquires the papilla recognition result 106 generated by the papilla recognition model 98. After the processing in step ST16 is executed, the medical support process proceeds to step ST18.

In step ST18, the recognition unit 80A determines whether or not the duodenal papilla 30A is shown in the frame 41 input to the papilla recognition model 98 in step ST14, based on the papilla recognition result 106 acquired in step ST16. In step ST18, in a case in which the duodenal papilla 30A is not shown in the frame 41 input to the papilla recognition model 98, a negative determination is made, and the medical support process proceeds to step ST42 shown in FIG. 11B. In step ST18, in a case in which the duodenal papilla 30A is shown in the frame 41 input to the papilla recognition model 98, an affirmative determination is made, and the medical support process proceeds to step ST20.

In step ST20, the recognition unit 80A inputs the frame 41 input to the papilla recognition model 98 in step ST14 to acquire the papilla recognition result 106 in step ST16, to the difficulty level recognition model 100. As a result, the difficulty level recognition model 100 generates the difficulty level recognition result 118. After the processing in step ST20 is executed, the medical support process proceeds to step ST22.

In step ST22, the recognition unit 80A acquires the difficulty level recognition result 118 generated by the difficulty level recognition model 100. After the processing in step ST22 is executed, the medical support process proceeds to step ST24.

In step ST24, the controller 80B determines whether or not the difficulty level Y included in the difficulty level recognition result 118 acquired in step ST22 is equal to or higher than the reference difficulty level. In step ST24, in a case in which the difficulty level Y included in the difficulty level recognition result 118 acquired in step ST22 is lower than the reference difficulty level, a negative determination is made, and the medical support process proceeds to step ST42 shown in FIG. 11B. In step ST24, in a case in which the difficulty level Y included in the difficulty level recognition result 118 acquired in step ST22 is equal to or higher than the reference difficulty level, an affirmative determination is made, and the medical support process proceeds to step ST26.

In step ST26, the controller 80B generates the high difficulty level notification information 126. The controller 80B displays the high difficulty level notification information 126 on the screen 36B as the text TX1. After the processing in step ST26 is executed, the medical support process proceeds to step ST28.

In step ST28, the recognition unit 80A determines whether or not the specific opening part is shown in the frame 41 input to the papilla recognition model 98 in step ST14 to acquire the papilla recognition result 106 in step ST16, based on the papilla recognition result 106 acquired in step ST16. In step ST28, in a case in which the specific opening part is not shown in the frame 41 input to the papilla recognition model 98 in step ST14 to acquire the papilla recognition result 106 in step ST16, a negative determination is made, and the medical support process proceeds to step ST42 shown in FIG. 11B. In step ST28, in a case in which the specific opening part is shown in the frame 41 input to the papilla recognition model 98 in step ST14 to acquire the papilla recognition result 106 in step ST16, an affirmative determination is made, and the medical support process proceeds to step ST30.

In step ST30, the recognition unit 80A acquires the confluence form information 120 received by the reception device 62. After the processing in step ST30 is executed, the medical support process proceeds to step ST32.

In step ST32, the recognition unit 80A inputs the frame 41 input to the papilla recognition model 98 in step ST14 to acquire the papilla recognition result 106 in step ST16, along with the confluence form information 120 acquired in step ST30, to the insertion direction prediction model 102. As a result, the insertion direction prediction model 102 generates the insertion direction information 122. After the processing in step ST32 is executed, the medical support process proceeds to step ST34.

In step ST34, the controller 80B acquires the insertion direction information 122 generated by the insertion direction prediction model 102 to which the frame 41 and the confluence form information 120 are input in step ST32. Then, the controller 80B displays, as the arrow marks 122A and 122B, the insertion direction information 122 in a superimposed manner on the frame 41 displayed on the screen 36A. After the processing in step ST34 is executed, the medical support process proceeds to step ST36 shown in FIG. 11B.

In step ST36, the controller 80B determines whether or not all of a first condition, a second condition, and a third condition are satisfied. The first condition refers to a condition in which the difficulty level Y included in the difficulty level recognition result 118 acquired in step ST22 is equal to or higher than the reference difficulty level. The second condition refers to a condition in which the specific opening part is shown in the frame 41 input to the papilla recognition model 98 in step ST14 to acquire the papilla recognition result 106 in step ST16. The third condition refers to a condition in which the confluence form specified from the confluence form information 120 acquired in step ST30 is the specific confluence form. In step ST36, in a case in which all of the first condition, the second condition, and the third condition are not satisfied, a negative determination is made, and the medical support process proceeds to step ST42. In step ST36, in a case in which all of the first condition, the second condition, and the third condition are satisfied, an affirmative determination is made, and the medical support process proceeds to step ST38.

In step ST38, the controller 80B acquires the support information 124 that meets the condition from the support information table 104. After the processing in step ST38 is executed, the medical support process proceeds to step ST40.

In step ST40, the controller 80B displays the support information 124 acquired in step ST38 on the screen 36B. After the processing in step ST40 is executed, the medical support process proceeds to step ST42.

In step ST42, the controller 80B determines whether or not a medical support process end condition is satisfied. An example of the medical support process end condition is a condition in which an instruction to end the medical support process is issued to the duodenoscope system 10 (for example, a condition in which the instruction to end the medical support process is received by the reception device 62).

In step ST42, in a case in which the medical support process end condition is not satisfied, a negative determination is made, and the medical support process proceeds to step ST10 shown in FIG. 11A. In a case in which the medical support process end condition is satisfied in step ST42, an affirmative determination is made, and the medical support process ends.

As described above, in the present embodiment, in a case in which the difficulty level Y determined based on the duodenal papilla 30A and the peripheral region 30B shown in the intestinal wall image 40 obtained by imaging the intestinal wall 30 via the duodenoscope 12 is equal to or higher than the reference difficulty level, the support information 124 is displayed on the screen 36B. Therefore, in a case in which the difficulty level Y of the intubation into the duodenal papilla 30A is equal to or higher than the reference difficulty level, the intubation into the duodenal papilla 30A can be supported. That is, since the support information 124 is displayed on the screen 36B in a case in which the difficulty level Y is equal to or higher than the reference difficulty level, the doctor 14 during the procedure can perform the intubation into the duodenal papilla 30A with high accuracy in a short time without determining whether or not the difficulty level Y of the intubation into the duodenal papilla 30A is equal to or higher than the reference difficulty level Y by himself/herself or performing an operation for displaying the support information 124 on the screen 36A and/or 36B.

In the present embodiment, the operation assistance information 124C is included in the support information 124 displayed on the screen 36B. Therefore, it is possible to make it easier for the doctor 14 during the procedure to perform an operation required for the intubation into the duodenal papilla 30A as the operation of the duodenoscope 12, as compared to a case in which the operation assistance information 124C is not displayed.

In addition, in the present embodiment, the difficulty level Y is determined based on the duodenal papilla 30A and the peripheral region 30B recognized by executing the recognition processing via AI (that is, the recognition processing using the papilla recognition model 98) on the intestinal wall image 40. Therefore, the difficulty level Y of the intubation into the duodenal papilla 30A can be determined with high accuracy as compared to a case in which the doctor 14 during the procedure visually specifies the duodenal papilla 30A and the peripheral region 30B to determine the difficulty level of the intubation into the duodenal papilla 30A.

In addition, in the present embodiment, in a case in which the difficulty level Y determined based on the duodenal papilla 30A and the peripheral region 30B shown in the intestinal wall image 40 obtained by imaging the intestinal wall 30 via the duodenoscope 12 is equal to or higher than the reference difficulty level, the insertion direction information 122 is displayed on the intestinal wall image 40 in a superimposed manner as the arrow marks 122A and 122B. Therefore, it is possible for the doctor 14 to accurately insert the cannula 54A into the bile duct 92A and the pancreatic duct 92B in a short time.

In addition, in the present embodiment, in a case in which the difficulty level Y determined based on the duodenal papilla 30A and the peripheral region 30B shown in the intestinal wall image 40 obtained by imaging the intestinal wall 30 via the duodenoscope 12 is equal to or higher than the reference difficulty level, the high difficulty level notification information 126 is displayed on the screen 36B. As a result, the doctor 14 during the procedure can visually recognize that the difficulty level Y of the intubation into the duodenal papilla 30A is equal to or higher than the reference difficulty level.

In addition, in the present embodiment, the difficulty level Y is determined based on the aspect of the duodenal papilla 30A. Therefore, the difficulty level of the intubation into the duodenal papilla 30A can be determined with high accuracy as compared to a case in which the difficulty level of the intubation into the duodenal papilla 30A is determined without considering the aspect of the duodenal papilla 30A.

In addition, in the present embodiment, the difficulty level Y is determined based on the shape of the duodenal papilla 30A. Therefore, the difficulty level of the intubation into the duodenal papilla 30A can be determined with high accuracy as compared to a case in which the difficulty level of the intubation into the duodenal papilla 30A is determined without considering the shape of the duodenal papilla 30A.

In addition, in the present embodiment, the difficulty level Y is determined based on the size of the duodenal papilla 30A. Therefore, the difficulty level of the intubation into the duodenal papilla 30A can be determined with high accuracy as compared to a case in which the difficulty level of the intubation into the duodenal papilla 30A is determined without considering the size of the duodenal papilla 30A.

In addition, in the present embodiment, the difficulty level Y is determined based on the type of the opening part 90A1 of the duodenal papilla 30A. Therefore, the difficulty level of the intubation into the duodenal papilla 30A can be determined with high accuracy as compared to a case in which the difficulty level of the intubation into the duodenal papilla 30A is determined without considering the type of the opening part 90A1 of the duodenal papilla 30A.

In addition, in the present embodiment, the difficulty level Y is determined based on the length of the oral side elevation of the duodenal papilla 30A. Therefore, the difficulty level of the intubation into the duodenal papilla 30A can be determined with high accuracy as compared to a case in which the difficulty level of the intubation into the duodenal papilla 30A is determined without considering the length of the oral side elevation of the duodenal papilla 30A.

In addition, in the present embodiment, the difficulty level Y is determined based on the aspect of the peripheral region 30B. Therefore, the difficulty level of the intubation into the duodenal papilla 30A can be determined with high accuracy as compared to a case in which the difficulty level of the intubation into the duodenal papilla 30A is determined without considering the aspect of the peripheral region 30B.

In addition, in the present embodiment, the difficulty level Y is determined based on the presence or absence of the diverticulum in the peripheral region 30B. Therefore, the difficulty level of the intubation into the duodenal papilla 30A can be determined with high accuracy as compared to a case in which the difficulty level of the intubation into the duodenal papilla 30A is determined without considering the presence or absence of the diverticulum in the peripheral region 30B.

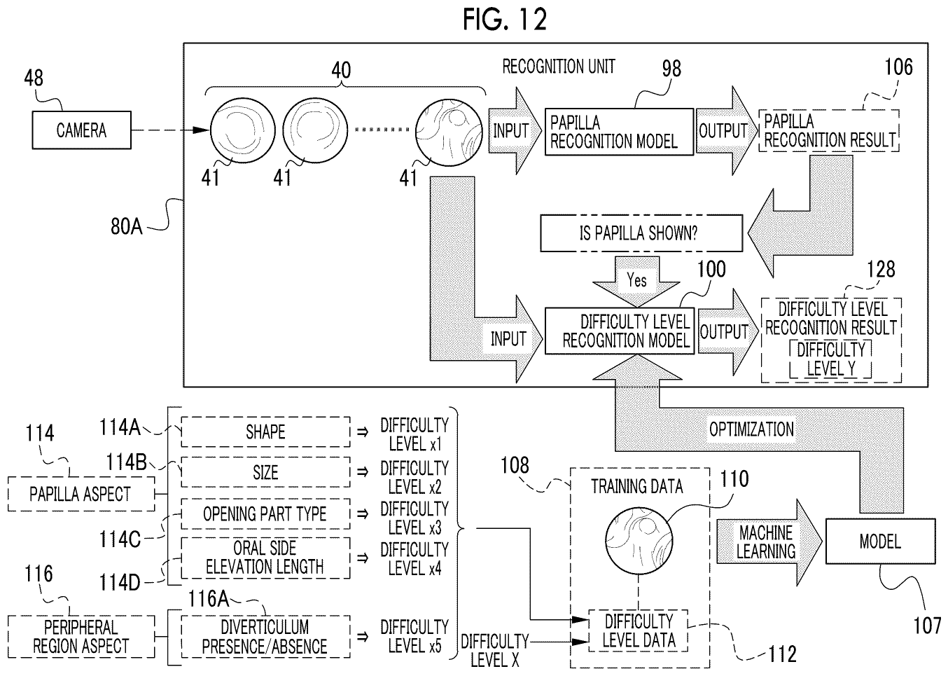

In the above-described embodiment, the data indicating the difficulty level X is described as an example of the difficulty level data 112 included in the training data 108 used in the machine learning for creating the difficulty level recognition model 100, but this is merely an example. For example, as shown in FIG. 12, the difficulty level data 112 may include the data indicating the difficulty level x1, the data indicating the difficulty level x2, the data indicating the difficulty level x3, the data indicating the difficulty level x4, and the data indicating the difficulty level x5, in addition to the data indicating the difficulty level X. In a case in which the difficulty level recognition model 100 is generated by executing the machine learning on the model 107 using the training data 108 including the difficulty level data 112 composed as described above, the difficulty level recognition model 100 generates a difficulty level recognition result 128, and outputs the generated difficulty level recognition result 128.

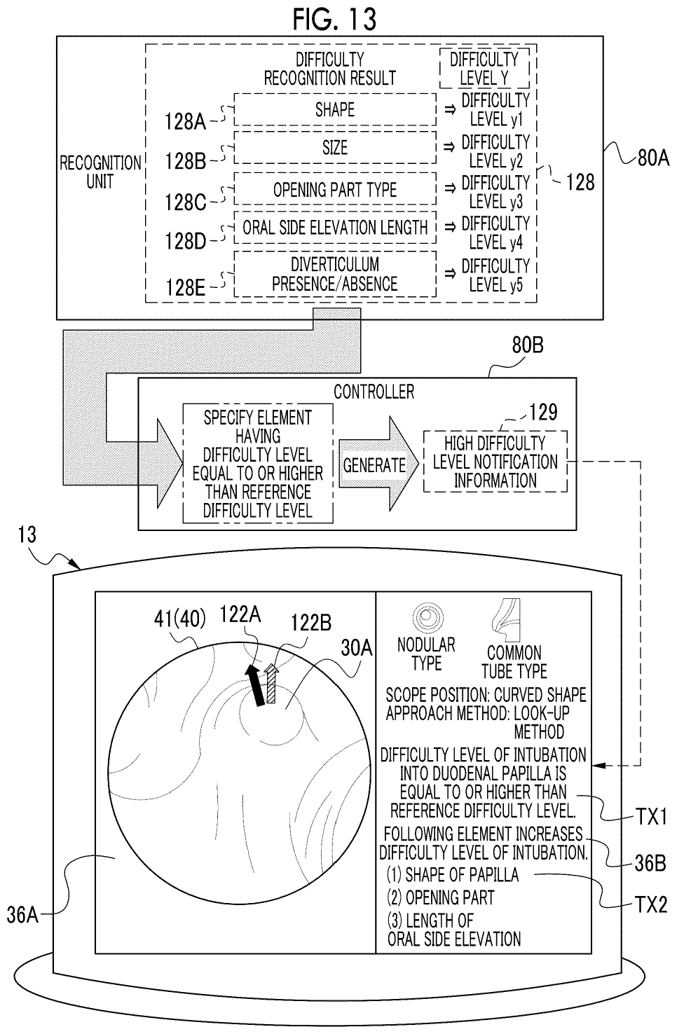

As shown in FIG. 13 as an example, the difficulty level recognition result 128 includes a difficulty level y1, a difficulty level y2, a difficulty level y3, a difficulty level y4, and a difficulty level y5, in addition to the difficulty level Y. The difficulty level Y included in the difficulty level recognition result 128 is a difficulty level comprehensively evaluated from the difficulty level y1, the difficulty level y2, the difficulty level y3, the difficulty level y4, and the difficulty level y5, as the difficulty level of the intubation into the duodenal papilla 30A. An example of the difficulty level comprehensively evaluated from the difficulty level y1, the difficulty level y2, the difficulty level y3, the difficulty level y4, and the difficulty level y5 is an average difficulty level of the difficulty level y1, the difficulty level y2, the difficulty level y3, the difficulty level y4, and the difficulty level y5.

The difficulty level y1 is a difficulty level corresponding to a shape 128A of the duodenal papilla 30A. That is, the difficulty level y1 means a difficulty level of the intubation into the duodenal papilla 30A in a case of the shape 128A. The difficulty level y2 is a difficulty level corresponding to a size 128B of the duodenal papilla 30A. That is, the difficulty level y2 means a difficulty level of the intubation into the duodenal papilla 30A in a case of the size 128B. The difficulty level y3 is a difficulty level corresponding to an opening part type 128C (that is, the type of the opening part 90A1 of the duodenal papilla 30A). That is, the difficulty level y3 means a difficulty level of the intubation into the duodenal papilla 30A in a case of the opening part type 128C. The difficulty level y4 is a difficulty level corresponding to an oral side elevation length 128D (that is, the length of the oral side elevation of the duodenal papilla 30A). That is, the difficulty level y4 means a difficulty level of the intubation into the duodenal papilla 30A in a case of the oral side elevation length 128D. The difficulty level y5 is a difficulty level corresponding to a diverticulum presence/absence 128E (that is, the presence or absence of the diverticulum). That is, the difficulty level y5 means a difficulty level of the intubation into the duodenal papilla 30A in a case of the diverticulum presence/absence 128E.

The controller 80B generates high difficulty level notification information 129 based on the difficulty level recognition result 128. The high difficulty level notification information 129 includes the high difficulty level notification information 126 according to the above-described embodiment and includes element specification information. The element specification information refers to information for specifying an element that increases the difficulty level of the intubation into the duodenal papilla 30A among a plurality of elements (here, as an example, the shape 128A, the size 128B, the opening part type 128C, the oral side elevation length 128D, and the diverticulum presence/absence 128E). Among the plurality of elements, an element that increases the difficulty level of the intubation into the duodenal papilla 30A is an element having a difficulty level equal to or higher than the reference difficulty level. The controller 80B specifies the element having the difficulty level equal to or higher than the reference difficulty level based on the difficulty level recognition result 128, generates the element specification information based on the specification result, and generates high difficulty level notification information 129 including the element specification information. The controller 80B displays the high difficulty level notification information 129 on the screen 36B in a text format. Accordingly, the text TX1 is displayed on the screen 36B and text TX2 is displayed on the screen 36B, as in the above-described embodiment. The text TX2 is text for specifying the element that increases the difficulty level of the intubation into the duodenal papilla 30A among the plurality of elements (here, as an example, the shape 128A, the size 128B, the opening part type 128C, the oral side elevation length 128D, and the diverticulum presence/absence 128E). Here, the text TX1 and the text TX2 are described as examples, but this is merely an example, and the visible display may be performed by using an image (for example, a mark) or the like instead of the text.

In the example shown in FIG. 13, the difficulty level Y is determined based on the shape 128A, the size 128B, the opening part type 128C, the oral side elevation length 128D, and the diverticulum presence/absence 128E, but this is merely an example. For example, the difficulty level Y may be determined based on the confluence form, which is one of the aspects of the duodenal papilla 30A, and on the deformation of the duodenum, which is one of the aspects of the peripheral region 30B. The deformation of the duodenum is caused by the medical treatment on the upper gastrointestinal tract (for example, resection of at least a part of the stomach).

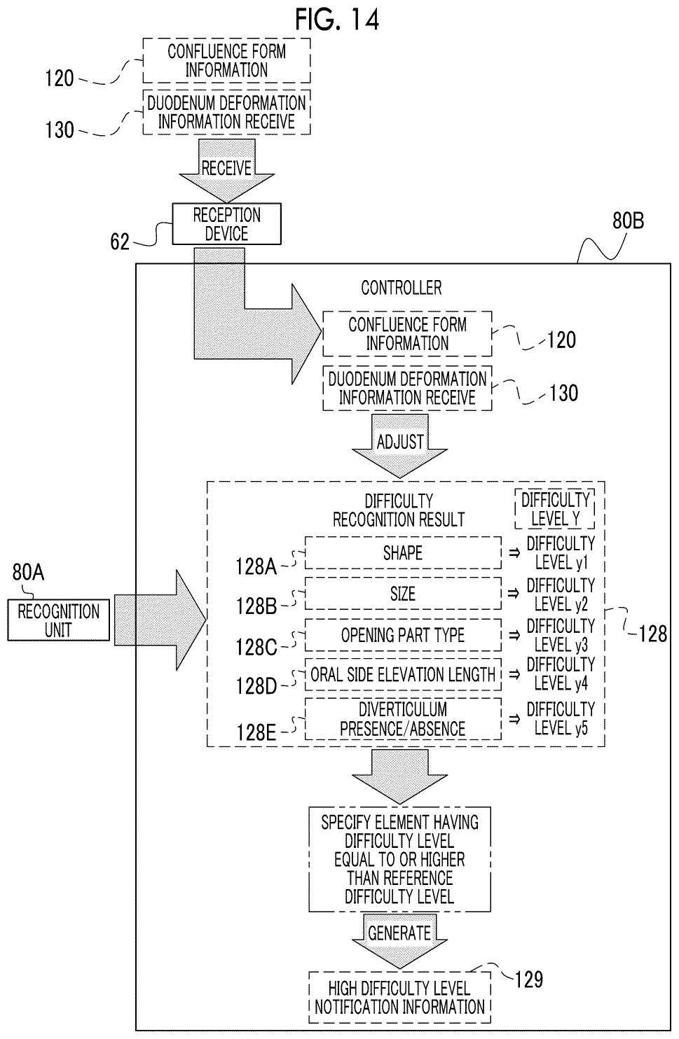

Examples of the determination of the difficulty level Y based on the confluence form and the deformation of the duodenum, as shown in FIG. 14, include an example in which the controller 80B adjusts the difficulty level recognition result 128 based on confluence form information 120 and duodenum deformation information 130. In the example shown in FIG. 14, the confluence form information 120 and the duodenum deformation information 130 are received by the reception device 62. The duodenum deformation information 130 includes information related to the duodenum deformation, such as information indicating that the duodenum is deformed, information indicating a degree of the deformation of the duodenum, and information for specifying a portion in which the deformation of the duodenum is equal to or more than a certain level.

The controller 80B adjusts the difficulty level y1, the difficulty level y2, the difficulty level y3, the difficulty level y4, and the difficulty level y5 in accordance with the confluence form information 120 and the duodenum deformation information 130. For example, the controller 80B adjusts the difficulty level y1, the difficulty level y2, the difficulty level y3, the difficulty level y4, and the difficulty level y5 by multiplying coefficients determined in accordance with the confluence form information 120 and the duodenum deformation information 130 by the difficulty level y1, the difficulty level y2, the difficulty level y3, the difficulty level y4, and the difficulty level y5. For example, the coefficients multiplied by the difficulty level y1, the difficulty level y2, the difficulty level y3, the difficulty level y4, and the difficulty level y5 need only be calculated from an arithmetic expression in which the confluence form information 120 and the duodenum deformation information 130 are independent variables and the coefficients are dependent variables. Here, although the form example is described in which both the confluence form information 120 and the duodenum deformation information 130 are used, this is merely an example, and one of the confluence form information 120 or the duodenum deformation information 130 may be used.

As described above, in the example shown in FIG. 14, since the difficulty level recognition result 128 is adjusted based on the confluence form, which is one of the aspects of the duodenal papilla 30A, the difficulty level Y of the intubation into the duodenal papilla 30A can be determined with high accuracy as compared to a case in which the difficulty level of the intubation into the duodenal papilla 30A is determined without considering the confluence form. In addition, in the example shown in FIG. 14, since the difficulty level recognition result 128 is adjusted based on the deformation of the duodenum, which is one of the aspects of the peripheral region 30B, the difficulty level Y of the intubation into the duodenal papilla 30A can be determined with high accuracy as compared to a case in which the difficulty level of the intubation into the duodenal papilla 30A is determined without considering the deformation of the duodenum.

In the example shown in FIG. 14, the form example is described in which the duodenum deformation information 130 received by the reception device 62 is acquired by the controller 80B, but this is merely an example. For example, in a process from the insertion of the duodenoscope 12 into the upper gastrointestinal tract to the duodenum, recognition processing using an AI method on the part of the upper gastrointestinal tract may be executed, and the duodenum deformation information 130 may be generated based on the recognition result of the part of the upper gastrointestinal tract (for example, non-recognition of the part, a certainty level lower than a threshold value, recognition of a deformed stomach, and/or recognition of a deformed duodenum).

In a case in which it is difficult to perform the intubation from the duodenal papilla 30A, the doctor 14 may incise the duodenal papilla 30A and expose the tube 92 from the duodenal papilla 30A. In this case, in order to implement high-accuracy incision of the duodenal papilla 30A, for example, information (for example, visible information such as a line) for specifying an incision region (that is, a region planned to be incised) for incising the duodenal papilla 30A may be displayed in a superimposed manner on the intestinal wall image 40 displayed on the screen 36A. In order to implement such superimposed display, it is important to predict the incision region for incising the duodenal papilla 30A first.

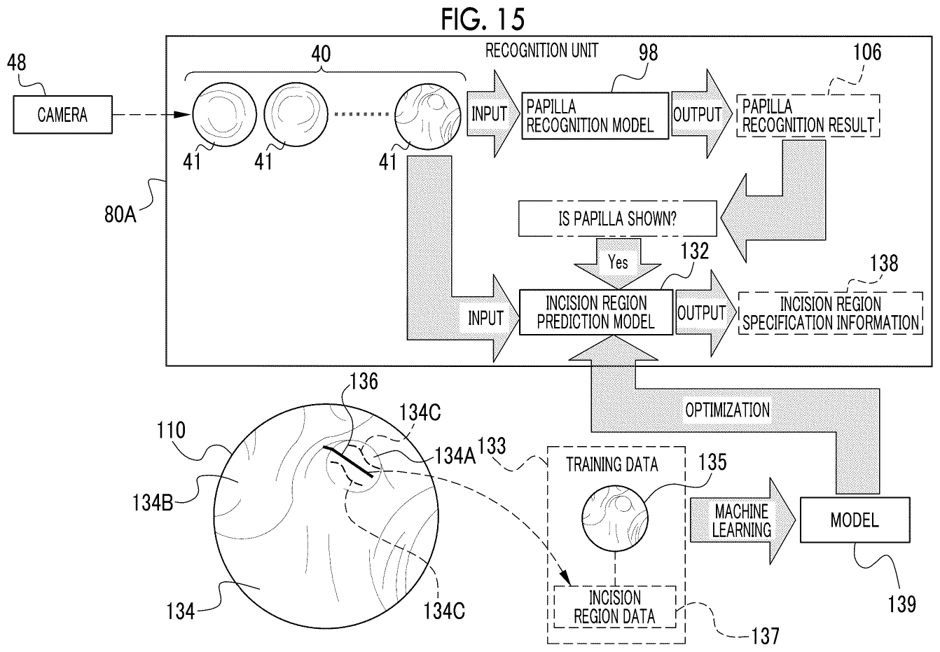

Therefore, in order to implement the prediction of the incision region, as an example, as shown in FIG. 15, in a case in which the duodenal papilla 30A and the peripheral region 30B are shown in the frame 41 input to the papilla recognition model 98 to obtain the papilla recognition result 106, the recognition unit 80A executes prediction processing using the AI method. The prediction processing using the AI method is implemented by image processing using an incision region prediction model 132. That is, the recognition unit 80A inputs the frame 41 input to the papilla recognition model 98 to obtain the papilla recognition result 106 to the incision region prediction model 132, to cause the incision region prediction model 132 to execute image processing. In the present embodiment, the incision region prediction model 132 is an example of a “first trained model” according to the present disclosure. In the present embodiment, the image processing using the incision region prediction model 132 is an example of “image processing” according to the present disclosure.

The incision region prediction model 132 is a trained model for prediction via AI, and is obtained by executing the machine learning for the frame 41. The incision region prediction model 132 has been optimized by training a neural network through machine learning using fourth training data that is a data set including a plurality of data (that is, data for a plurality of frames) in which fourth example data and fourth correct answer data are associated with each other. That is, the insertion direction prediction model 102 is a trained model that has been optimized to receive input of fourth example data to generate fourth correct answer data.

In the example shown in FIG. 15, training data 133 is shown as the fourth training data. The training data 133 includes a sample image 135 as an example of the fourth example data, and incision region data 137 as an example of fourth correct answer data, and the sample image 135 and the incision region data 137 are associated with each other.

The sample image 135 is the same image as the image used in the above-described first example data. An intestinal wall 134 of the duodenum, a duodenal papilla 134A, and a peripheral region 134B of the duodenal papilla 134A are shown in the sample image 135.

The incision region data 137 is data for specifying an incision region 136 for incising the duodenal papilla 134A. The data for specifying the incision region 136 is data (for example, coordinates) for specifying the geometrical characteristics (for example, the position, the size, and the shape) of the incision region 136 in the sample image 135.

The incision region 136 is included in a region avoiding a plurality of blood vessels 134C in the duodenal papilla 134A. The plurality of blood vessels 134C are blood vessels that are included in the duodenal papilla 134A and that have an attention level equal to or higher than a certain attention level. A certain attention level means an attention level to a thinnest blood vessel in which the tube (for example, the bile duct and/or the pancreatic duct) is expected to be invisible due to bleeding associated with damage to the blood vessel. In a case in which the sample image 135 is an image obtained by imaging the reflected light obtained by irradiating the intestinal wall 134 with the special light, a presence position of the blood vessel that is included in the duodenal papilla 134A and that has the attention level equal to or higher than a certain attention level is visually specified by the annotator.

The incision region prediction model 132 has been optimized by executing the machine learning using the training data 133 on a model 139 (for example, a neural network).

The recognition unit 80A inputs the frame 41 to the incision region prediction model 132 at the frame rate at which the frame 41 is displayed on the screen 36A. Examples of the frame 41 input to the incision region prediction model 132 include an image obtained by imaging the reflected light obtained by irradiating the intestinal wall 30 with the special light. The image obtained by imaging the reflected light obtained by irradiating the intestinal wall 30 with the special light is an image in which the blood vessel that is included in the duodenal papilla 30A and that has the attention level equal to or higher than a certain attention level is shown in a state of being specifiable. In the example shown in FIG. 15, the frame 41 input to the incision region prediction model 132 is an example of a “first intestinal wall image” according to the present disclosure.

The incision region prediction model 132 predicts the incision region (that is, the region planned to be incised) for incising the duodenal papilla 30A shown in the input frame 41 each time the frame 41 is input. The incision region for incising the duodenal papilla 30A is expected to be within the region avoiding the blood vessel that is included in the duodenal papilla 30A and that has the attention level equal to or higher than a certain attention level by executing the image processing using the incision region prediction model 132 on the frame 41. The incision region prediction model 132 generates incision region specification information 138 as a prediction result. The incision region specification information 138 refers to information for specifying the incision region for incising the duodenal papilla 30A. In the present embodiment, the incision region specification information 138 is an example of “incision region specification information” and “support information” according to the present disclosure.

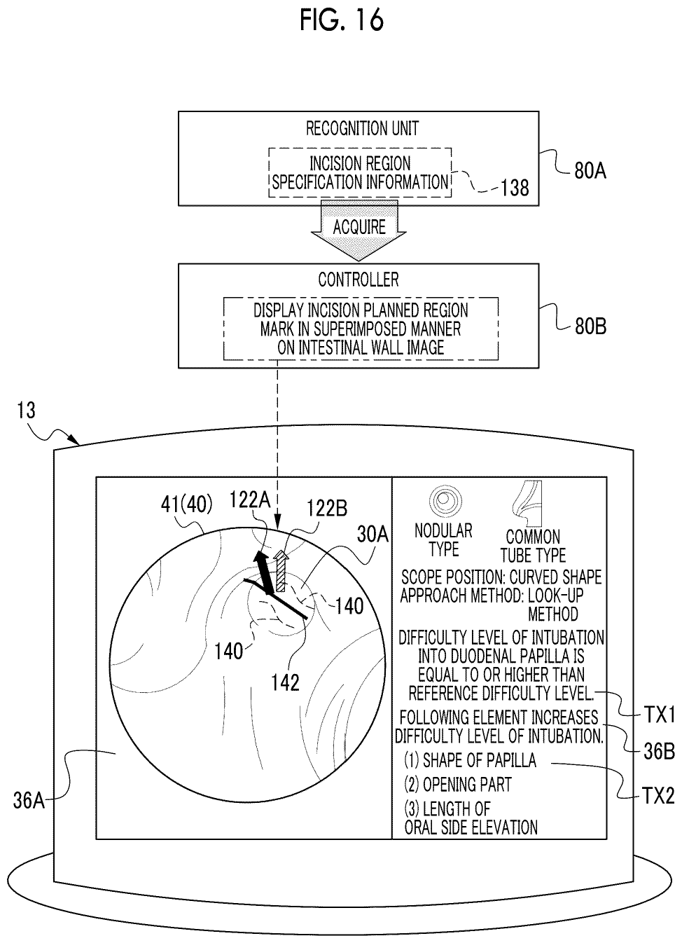

As shown in FIG. 16 as an example, the controller 80B acquires the incision region specification information 138 from the recognition unit 80A. The controller 80B displays the incision region specification information 138 in a superimposed manner on the frame 41 displayed on the screen 36A in a display aspect that is distinguishable from other image regions as an incision planned region mark 142, which is a mark for specifying the incision region. In the example shown in FIG. 16, the incision planned region mark 142 is a linear mark. The incision region specified by the incision planned region mark 142 is included in a region avoiding a plurality of blood vessels 140 included in the duodenal papilla 30A. The plurality of blood vessels 140 are blood vessels that are included in the duodenal papilla 30A and that have the attention level equal to or higher than a certain attention level. In the present embodiment, the plurality of blood vessels 140 are an example of a “blood vessel that is included in the duodenal papilla and that has an attention level equal to or higher than a certain attention level” according to the present disclosure.

As described above, in the examples shown in FIGS. 15 and 16, the incision region specification information 138 is displayed in a superimposed manner on the frame 41 displayed on the screen 36A in a display aspect that is distinguishable from other image regions as the incision planned region mark 142, and thus the doctor 14 can incise the duodenal papilla 30A with high accuracy as compared to a case in which the incision region specification information 138 is not displayed.

In addition, in the example shown in FIG. 15, the incision region is predicted by the incision region prediction model 132 in a region avoiding the plurality of blood vessels 140 that are included in the duodenal papilla 30A and that have the attention level equal to or higher than a certain attention level. Therefore, the doctor 14 can incise the duodenal papilla 30A without damaging the blood vessel 140.

In addition, in the example shown in FIG. 15, the incision region is predicted by executing the image processing using the incision region prediction model 132 on the frame 21, and the incision region specification information 138 is obtained as the prediction result obtained by the image processing. Therefore, the doctor 14 can specify the incision region with high accuracy as compared to a case in which the doctor 14 specifies the incision region only through visual observation.

In addition, in the example shown in FIG. 15, an image in which the blood vessel that is included in the duodenal papilla 30A and that has the attention level equal to or higher than a certain attention level is shown in a state of being specifiable is used as the frame 21 input to the incision region prediction model 132. Therefore, the doctor 14 can specify the incision region avoiding the blood vessel that is included in the duodenal papilla 30A and that has the attention level equal to or higher than a certain attention level, with high accuracy as compared to a case in which the doctor 14 specifies the incision region only through visual observation.

In the above-described embodiment, the form example is described in which the difficulty level data 112 is determined based on the papilla aspect 114 and the peripheral region aspect 116, but this is merely an example, and the difficulty level data 112 may be determined based on one of the papilla aspect 114 or the peripheral region aspect 116.

In the above-described embodiment, the form example is described in which the shape 114A, the size 114B, the opening part type 114C, and the oral side elevation length 114D are included in the papilla aspect 114, but this is merely an example, and the papilla aspect 114 need only include at least one of the shape 114A, the size 114B, the opening part type 114C, or the oral side elevation length 114D.

In the above-described embodiment, the description is made on the premise that the doctor 14 completes the intubation into the duodenal papilla 30A, but in a case in which it is recognized by the AI method recognition processing that the intubation into the duodenal papilla 30A is not completed even after a certain time (for example, 10 minutes) or more has elapsed, the processor 80 may display information prompting a change of an operator and/or information prompting a change of a surgical method on the screen 36A and/or 36B. Further, the information for prompting the change of the operator and/or the information for prompting the change of the surgical method may be output by voice.

In the above-described embodiment, the form example is described in which the insertion direction information 122, the support information 124, and the high difficulty level notification information 126 are displayed as the visible information, but this is merely an example, and at least a part of the insertion direction information 122 and the support information 124 and/or the high difficulty level notification information 126 may be output by voice.

In the above-described embodiment, the trained model for object recognition using the segmentation method via the AI is described as the papilla recognition model 98, but the present disclosure is established even in a case in which a trained model for object recognition using a bounding box method via AI is used.

In the above-described embodiment, the form example is described in which the insertion direction information 122 is displayed in a superimposed manner on the frame 21 as the visible information, but the present disclosure is not limited to this. For example, the position of the opening part 90A1 may be specified by the image processing using the papilla recognition model 98, and an image (for example, a mark) for specifying the position of the opening part 90A1 may be displayed in a superimposed manner on the frame 21.

In the above-described embodiment, as an example of the confluence form information 120, the information indicating the confluence form that is specified in advance from the examination result is described, but the present disclosure is not limited to this. For example, the confluence form may be specified by the doctor 14 through visual observation from the image obtained by the X-ray imaging during the ERCP examination, or the confluence form may be specified by executing image analysis processing via the computer 76 or the like on the image obtained by the X-ray imaging during the ERCP examination, to generate the confluence form information 120 based on the specification result.

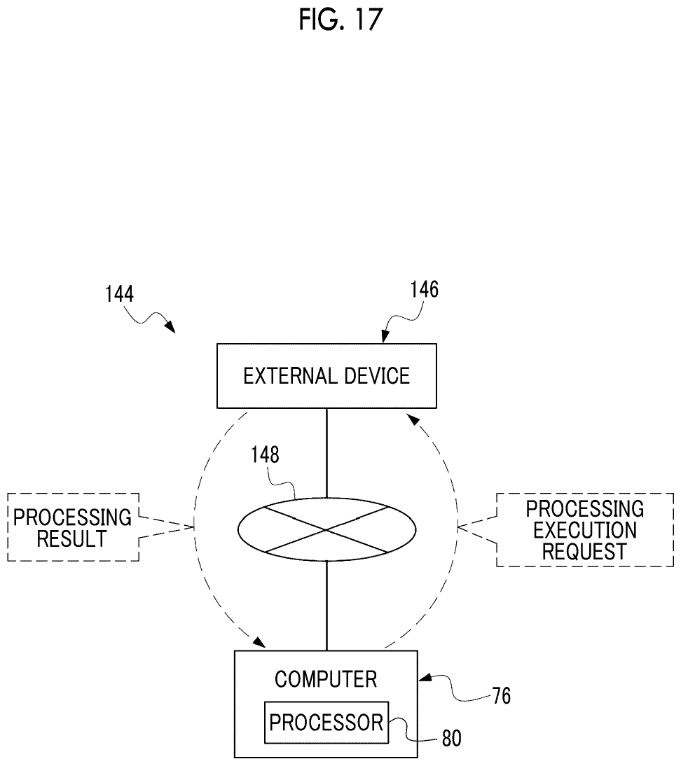

In the above-described embodiment, the form example is described in which the medical support process is executed by the computer 76, but the present disclosure is not limited thereto, and at least a part of the processing included in the medical support process may be executed by a device provided outside the computer 76. Hereinafter, an example of such a case will be described with reference to FIG. 17.

FIG. 17 is a conceptual diagram showing an example of a configuration of a duodenoscope system 144. The duodenoscope system 144 is an example of an “endoscope system” according to the present disclosure. The duodenoscope system 144 differs from the duodenoscope system 10 according to the above-described embodiment in that an external device 146 is provided.

The external device 146 is connected communicably to the computer 76 via a network 148 (for example, a WAN and/or a LAN).

Examples of the external device 146 include at least one server that directly or indirectly transmits and receives data to and from the computer 76 via the network 148. The external device 146 receives a processing execution instruction issued from the processor 80 of the computer 76 via the network 148. The external device 146 executes processing corresponding to the received processing execution instruction and transmits a processing result to the computer 76 via the network 148. In the computer 76, the processor 80 receives the processing result transmitted from the external device 146 via the network 148, and executes processing using the received processing result.

Examples of the processing execution instruction include an instruction to cause the external device 146 to execute at least a part of the medical support process. A first example of at least a part of the medical support process (that is, the processing executed by the external device 146) is AI processing using the papilla recognition model 98, the difficulty level recognition model 100, the insertion direction prediction model 102, and/or the incision region prediction model 132. In this case, the external device 146 executes the AI processing in response to the processing execution instruction given from the processor 80 via the network 148, and transmits the AI processing result to the computer 76 via the network 148. In the computer 76, the processor 80 receives the AI processing result and executes the same processing as in the above-described embodiment by using the received AI processing result.

A second example of at least a part of the medical support process (that is, the processing executed by the external device 146) is partial processing executed by the controller 80B. Examples thereof include processing of acquiring the support information 124 from the support information table 104, processing of executing various determinations, and/or processing of generating information. In this case, the external device 146 executes the processing of acquiring the support information 124 from the support information table 104, the processing of executing various determinations, and/or the processing of generating information in response to the processing execution instruction given from the processor 80 via the network 148, and transmits the processing result to the computer 76 via the network 148. In the computer 76, the processor 80 receives the processing result to execute the same processing as in the above-described embodiment by using the received processing result.

For example, the external device 146 is implemented by cloud computing. It should be noted that the cloud computing is merely an example, and the external device 146 may be implemented by network computing such as fog computing, edge computing, or grid computing. Instead of the server, at least one personal computer or the like may be used as the external device 146. In addition, an operation device having a communication function equipped with a plurality of types of AI functions may be used.

In the above-described embodiment, the form example is described in which the medical support program 96 is stored in the storage 84, but the present disclosure is not limited thereto. For example, the medical support program 96 may be stored in a portable computer-readable non-transitory storage medium such as an SSD or a USB memory. The medical support program 96 stored in the non-transitory storage medium is installed in the computer 76 of the duodenoscope system 10. The processor 80 executes the medical support process in accordance with the medical support program 96.

In addition, the medical support program 96 may be stored in a storage device of another computer or server connected to the duodenoscope system 10 via a network, and the medical support program 96 may be downloaded in response to a request from the duodenoscope system 10 and installed in the computer 76.

It should be noted that it is not necessary to store the entire medical support program 96 in a storage device of another computer or server connected to the duodenoscope system 10 or to store the entire medical support program 96 in the storage 84, and a part of the medical support program 96 may be stored.

Various processors described below can be used as a hardware resource for executing the medical support process. Examples of the processor include a CPU which is a general-purpose processor that functions as the hardware resource executing the medical support process by executing software, that is, a program. In addition, examples of the processor include a dedicated electric circuit which is a processor of which a circuit configuration is specially designed for executing specific processing, such as an FPGA, a PLD, or an ASIC. Any processor has a memory built in or connected to it, and any processor executes the medical support process by using the memory.

The hardware resource for executing the medical support process may be configured by one of the various processors or by combining two or more processors of the same type or different types (for example, by combining a plurality of FPGAs or by combining a CPU and an FPGA). The hardware resource for executing the medical support process may also be one processor.

As an example of the configuration using one processor, first, there is a form in which one processor is configured by combining one or more CPUs and software, and the processor functions as the hardware resource for executing the medical support process. As a second example, as typified by an SoC or the like, there is a form in which a processor that implements all functions of a system including a plurality of hardware resources executing the medical support process with one IC chip is used. As described above, the medical support process is implemented by using one or more of the various processors as the hardware resource.

As a hardware structure of these various processors, more specifically, an electric circuit in which circuit elements, such as semiconductor elements, are combined can be used. Further, the medical support process is merely an example. Therefore, it goes without saying that unnecessary steps may be deleted, new steps may be added, or the processing order may be changed within a range that does not deviate from the gist.

The above-described contents and the above-shown contents are the detailed description of the parts according to the present disclosure, and are merely examples of the present disclosure. For example, the description of the configuration, the function, the operation, and the effect is the description of examples of the configuration, the function, the operation, and the effect of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. In addition, in order to avoid complications and facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which the individual documents, patent applications, and technical standards are specifically and individually stated to be described by reference.

What is claimed is:

1. A medical support device comprising:
a processor,
wherein the processor is configured to:
acquire a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope, and
output support information for supporting the intubation in a case in which the difficulty level is equal to or higher than a reference difficulty level.

2. The medical support device according to claim 1,
wherein, in a case in which the difficulty level is determined based on the duodenal papilla,
the difficulty level is determined based on an aspect of the duodenal papilla.

3. The medical support device according to claim 2,
wherein the aspect of the duodenal papilla includes a shape of the duodenal papilla.

4. The medical support device according to claim 2,
wherein the aspect of the duodenal papilla includes a size of the duodenal papilla.

5. The medical support device according to claim 2,
wherein the aspect of the duodenal papilla includes a type of an opening part of the duodenal papilla.

6. The medical support device according to claim 2,
wherein the aspect of the duodenal papilla includes a confluence form of a bile duct and a pancreatic duct in the duodenal papilla.

7. The medical support device according to claim 2,
wherein the aspect of the duodenal papilla includes a length of an oral side elevation.

8. The medical support device according to claim 1,
wherein, in a case in which the difficulty level is determined based on the peripheral region,
the difficulty level is determined based on an aspect of the peripheral region.

9. The medical support device according to claim 8,
wherein the aspect of the peripheral region includes presence or absence of a diverticulum.

10. The medical support device according to claim 8,
wherein the aspect of the peripheral region includes deformation of the duodenum.

11. The medical support device according to claim 1,
wherein the support information includes information indicating that the difficulty level is equal to or higher than the reference difficulty level.

12. The medical support device according to claim 1,
wherein the support information includes incision region specification information for specifying an incision region for incising the duodenal papilla.

13. The medical support device according to claim 12,
wherein the incision region is included in a region avoiding a blood vessel that is included in the duodenal papilla and that has an attention level equal to or higher than a certain attention level.

14. The medical support device according to claim 12,
wherein the incision region is predicted by executing image processing on the intestinal wall image, and
the incision region specification information is a prediction result of the incision region obtained by the image processing.

15. The medical support device according to claim 14,
wherein the image processing is processing of causing a first trained model to generate the prediction result by inputting the intestinal wall image to the first trained model.

16. The medical support device according to claim 14,
wherein the intestinal wall image includes a first intestinal wall image in which a blood vessel that is included in the duodenal papilla and that has an attention level equal to or higher than a certain attention level is shown in a state of being specifiable, and
the incision region is predicted to be within a region avoiding the blood vessel by executing the image processing on the first intestinal wall image.

17. The medical support device according to claim 1,
wherein the support information includes operation assistance information for assisting an operation of the endoscope.

18. The medical support device according to claim 1,
wherein the difficulty level is determined based on the duodenal papilla and/or the peripheral region recognized by executing recognition processing on the intestinal wall image.

19. The medical support device according to claim 18,
wherein the recognition processing is processing of causing a second trained model to recognize the duodenal papilla and/or the peripheral region shown in the intestinal wall image by inputting the intestinal wall image to the second trained model.

20. An endoscope system comprising:
the medical support device according to claim 1; and
the endoscope.

21. A medical support method comprising:
acquiring a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope; and
outputting support information for supporting the intubation in a case in which the difficulty level is equal to or higher than a reference difficulty level.

22. A non-transitory computer-readable storage medium storing a program executable by a computer to execute a medical support process comprising:
acquiring a difficulty level of intubation into a duodenal papilla, the difficulty level being determined based on the duodenal papilla and/or a peripheral region of the duodenal papilla, which is shown in an intestinal wall image obtained by imaging an intestinal wall of a duodenum via an endoscope; and
outputting support information for supporting the intubation in a case in which the difficulty level is equal to or higher than a reference difficulty level.
